(19) United States
(12) Patent Application Publication
(10) Pub. No.: US 2008/0081835 A1
(43) Pub. Date: Apr. 3, 2008

(54) Use of Ltb4 Inhibitors for the Treatment of B-Cell Leukemias and Lymphomas

(76) Inventors: Hans-Erik Claesson, Stockholm (SE); Magnus Bjorkholm, Stockholm (SE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/579,474

(22) PCT Filed: May 5, 2005
(86) PCT No.: PCT/GB2005/01724

§ 371 Date: Mar. 14, 2007

Related U.S. Application Data
(60) Provisional application No. 60/568,262, filed on May 6, 2004.

(30) Foreign Application Priority Data
May 6, 2004 (GB) 0410103.6

(51) Int. Cl. A61K 31/404; A61P 35/00

(52) U.S. Cl. 514/418

(57) ABSTRACT

The invention relates to the use of an inhibitor of the biosynthesis and/or function of LTB₄ for the manufacture of a medicament for the treatment of B-cell chronic lymphocytic leukemia (B-CLL), B-prolymphocytic leukemia (B-PLL) or B-cell lymphoma. Preferably, the inhibitor of the biosynthesis and/or function of LTB₄ is the inhibitor of 5-LO BWA4C or the inhibitor of FLAP MK-886.

### FIELD OF THE INVENTION

This invention relates to a method of treating B-cell chronic lymphocytic leukemia (B-CLL), B-Prolymphocytic leukemia (B-PLL) or B-cell lymphoma (non-Hodgkin lymphoma, NHL), which method utilises inhibitors of the biosynthesis and/or function of LTB₄ (e.g. inhibitors of leukotriene B₄ (LTB₄) biosynthesis and/or antagonists of the BLT1 receptor).

### BACKGROUND AND PRIOR ART

Leukotrienes (LTs) are biologically active metabolites of arachidonic acid. Once liberated by phospholipase A₂ (E.C.3.1.1.4), arachidonic acid can be converted to prostaglandins, thromboxanes, and leukotrienes. The key enzyme in leukotriene biosynthesis is 5-lipoxygenase (5-LO) (E.C.1.13.11.34), which in a two-step reaction catalyzes the formation of leukotriene As (LTA₄) from arachidonic acid. LTA₄ can be further metabolized into leukotriene B₄ (LTB₄), a reaction catalyzed by LTA₄ hydrolase (E.C.3.3.2.6). Cellular leukotriene biosynthesis is dependent on 5-lipoxygenase activating protein (FLAP), a membrane bound protein which binds arachidonic acid and facilitates the 5-lipoxygenase reaction.

In contrast to prostaglandins, which are produced by almost all type of cells, formation of leukotrienes from arachidonic acid is restricted to a few cell types in the human body. Biosynthesis of leukotrienes occurs mainly in myeloid cells and B-lymphocytes. The production of LTB₄ and the biological effects of this compound on myeloid cells are well characterized, and LTB₄ stimulates neutrophil trafficking and activation at very low concentrations.

However, the biosynthesis and function of leukotrienes by B-lymphocytes are much less well characterized. In contrast to myeloid cells, intact B cells do not produce LTB₄ after challenge with calcium ionophore A23187 only. The mechanism of activation of leukotriene biosynthesis in intact B cells is unclear, but there is accumulating evidence that the cellular redox status is an important parameter for biosynthesis of leukotrienes. Furthermore, the p38 mitogen-activated protein kinase appears also to be involved in stress-induced leukotriene synthesis in B-cells.

There is no convincing report demonstrating that T lymphocytes contain 5-lipoxygenase and can produce leukotrienes. However, T lymphocytes express FLAP but the function of this protein in T cells is not known.

The actions of LTB₄ on leukocytes are mainly mediated by BLT1, a high-affinity G-coupled LTB₄ receptor expressed on neutrophils and monocytes. BLT1 is also expressed on activated T lymphocytes, both cytotoxic CD8+ cells and CD4+ cells and weakly on peripheral human non-activated B-lymphocytes. A second LTB₄ receptor with lower substrate affinity and wider tissue distribution has also been characterized.

LTB₄ is an immunomodulator and this compound activates B cells, T cells and NK cells (see Int. J. Immunopharmacol. 14, 441 (1992)). LTB₄ enhances activation, proliferation and antibody production in tonsillar B lymphocytes (see: J. Immunol. 143, 1996 (1989); Cell Immunol. 156, 124 (1994); and J. Immunol. 145, 3406 (1990)) and stimulates various T-cell functions. LTB₄ is a very potent chemotactic compound for activated T lymphocytes and BLTL1-receptor deficient mice have an impaired trafficking of activated CD8⁺ cells and CD4⁺ cells. Furthermore, L-TB₄ enhances also NK cell activity and cytotoxic T cell function.

B-Chronic lymphocytic leukemia (B-CLL) represents the most frequent leukemia of adults, having an incidence of 3 per 100,000 per year in the western hemisphere. Treatment regimes for B-CLL vary with the stage of progression of the disease. Current treatments for advanced B-CLL include chlorambucil, purine analogues (e.g. fludarabine), monoclonal antibodies (e.g. alemtuzumab and rituximab), and combinations of fludarabine with other chemotherapeutics (e.g. cyclophosphamide, chlorambucil or rituximab).

B-Prolymphocytic leukemia (B-PLL) is a rare form of leukemia, usually seen in elderly men, and treated with chemotherapeutic agents. However, the prognosis for patients with B-PLL is poor, as most die within 48 months of diagnosis

Lymphomas (Hodgkin's and non-Hodgkin lymphoma; HL and NHL) constitute the largest group of hematological malignancies. Treatment options include watch-and-wait (patients with indolent NHL), radiation (limited disease), chemotherapy (the large majority of patients will be exposed to combination chemotherapy), biologic therapy, and stem cell/bone marrow transplant. For aggressive NHL, CHOP in combination with rituximab (monoclonal antibody directed against the CD20 antigen) sometimes with the addition of etoposide (younger patients) and often with granulocyte colony stimulating factor support is prevailing.

However, the leukemias mentioned above remain incurable. Moreover, despite obvious therapeutic progress, most patients having B-cell lymphoma die from their disease or from treatment-related complications. Thus, there is a need for further chemotherapeutic agents that are capable of treating B-CLL, B-PLL and/ or B-cell lymphoma.

Agents that block lipoxygenase-catalysed activity are known to be potentially useful as cancer chemopreventatives (see, for example, Cancer Epidemiology, Biomarkers & Prevention 8, 467 (1998)).

Indeed, MK-886, an inhibitor of FLAP has been observed to have antiproliferative effects against human lung cancer cells and malignant cells from patients with acute or chronic myelogenous leukemia (see J. Clin. Invest. 97, 806 (1996), Anticancer Res. 16, 2589 (1996), Leukemia Res. 22(1), 49 (1998) and Leukemia Res. 17(9), 759 (1993)).

However, as mentioned above, the function of leukotrienes in B-lymphocytes is not well understood, and B-cells differ substantially from myeloid cells in respect of the conditions under which they produce LTB₄. Thus, to the applicant's knowledge, none of the above-mentioned documents disclose or suggest the use of inhibitors of the biosynthesis and/or function of LTB₄ in the treatment of B-CLL, B-PLL or B-cell lymphoma.

### SUMMARY OF THE INVENTION

We have found, surprisingly, that inhibitors of the biosynthesis and/or function of LTB₄ have antiproliferative effects on B-cells from patients suffering with B-CLL, and hence have utility in the treatment of B-CLL, B-PLL or B-cell lymphoma.

Therefore, according to a first aspect of the invention there is provided a method of treating B-CLL, B-PLL or B-cell lymphoma, which method comprises administering an inhibitor of the biosynthesis and/or function of LTB₄ to a patient in need of such treatment.

According to a second aspect of the invention, there is provided the use of an inhibitor of the biosynthesis and/or function of LTB₄ in the preparation of a medicament for the treatment of B-CLL, B-PLL or B-cell lymphoma.

The treatment of B-CLL, B-PLL or B-cell lymphoma may be effected by co-administration of cancer chemotherapeutic agents that are not inhibitors of the biosynthesis and/or function of LTB₄ (i.e. agents that have a different mechanism of action in treating B-CLL, B-PLL or B-cell-lymphoma).

In this respect, according to a third aspect of the invention, there is provided a method of treating B-CLL, B-PLL or B-cell lymphoma, which method comprises administering an inhibitor of the biosynthesis and/or function of LTB₄ to a patient in need of such treatment, which patient is administered a cancer chemotherapeutic agent having a different mechanism of action.

Also, according to a fourth aspect of the invention, there is provided the use of an inhibitor of the biosynthesis and/or function of LTB₄ in the preparation of a medicament for the treatment of B-CLL, B-PLL or B-cell lymphoma in a patient who is administered a cancer chemotherapeutic agent having a different mechanism of action.

Conversely, according to fifth and sixth aspects of the invention, respectively, there is provided:
(a) a method of treating B-CLL, B-PLL or B-cell lymphoma, which method comprises administering, as sole cancer chemotherapeutic agent, an inhibitor of the biosynthesis and/or function of LTB₄ to a patient in need of such treatment; and
(b) the use of an inhibitor of the biosynthesis and/or function of LTB₄ as the sole cancer chemotherapeutic agent in the preparation of a medicament for the treatment of B-CLL, B-PLL or B-cell lymphoma.

Furthermore, according to seventh aspect of the invention, there is provided a combination product comprising:
(A) an inhibitor of the biosynthesis and/or function of LTB₄, or a pharmaceutically-acceptable derivative thereof, and
(B) a cancer chemotherapeutic agent having a different mechanism of action, or a pharmaceutically acceptable derivative thereof,
   wherein each of components (A) and (B) is formulated in admixture with a pharmaceutically-acceptable adjuvant, diluent or carrier.

Such combination products may be presented either as separate formulations, wherein at least one of those formulations comprises an inhibitor of the biosynthesis and/or function of LTB₄/derivative and at least one comprises the other cancer chemotherapeutic therapeutic agent, or may be presented (i.e. formulated) as a combined preparation (i.e. presented as a single formulation including components (A) and (B)).

In a particular embodiment of this aspect of the invention, component (A) is an inhibitor of the biosynthesis of LTB₄, or a pharmaceutically-acceptable derivative thereof.

### Definitions

When used herein, the term “inhibitor of the biosynthesis of LTB₄” includes references to inhibitors of 5-LO, inhibitors of FLAP and/or inhibitors of leukotriene A₄ (LTA₄) hydrolase. Preferred inhibitors of the biosynthesis of LTB₄ include inhibitors of 5-LO and inhibitors of FLAP, such as the specific inhibitors mentioned below (and particularly the 5-LO inhibitor BWA4C and/or the FLAP inhibitor MK-886).

In this respect, inhibitors of the biosynthesis of LTB₄ may or may not be BWA4C or MK-886.

When used herein, the term “inhibitor of the function of LTB₄” includes references to compounds that antagonise the receptors for LTB₄, such as antagonists of the BLT1 receptor.

Thus, according to a preferred embodiment of the invention, the method of treating B-CLL, B-PLL or B-cell lymphoma comprises administering inhibitor of the biosynthesis of LTB₄ and/or an antagonist of the BLT1 receptor to a patient in need of treatment for B-CLL, B-PLL or B-cell lymphoma.

Further, according to a more preferred embodiment of the invention, the method of treating B-CLL, B-PLL or B-cell lymphoma comprises administering an inhibitor of the biosynthesis of LTB₄ (such a 5-LO and/or a FLAP inhibitor) to a patient in need of treatment for B-CLL, B-PLL or B-cell lymphoma. In a particularly preferred embodiment, the method of the invention comprises administering to the patient an inhibitor of 5-LO (e.g. BWA4C) or an inhibitor of FLAP (e.g. MK-886).

Whether a compound is an inhibitor of 5-LO, FLAP and/or LTA₄ hydrolase, and/or an antagonist of the BLT1 receptor may be determined by techniques know to those skilled in the art. For example:
(i) inhibition of 5-LO may be determined in sonicated leukocytes incubated with arachidonic acid;
(ii) inhibition of FLAP may be determined by monitoring intact leukocytes that have been stimulated with calcium ionophore A23187 (the inhibitor should not block the formation of leukotrienes in sonicated cells incubated with arachidonic acid);
(iii) inhibition of LTA₄ hydrolase may be determined by monitoring the metabolism of synthetic LTA₄ in either whole cells or with purified LTA₄ hydrolase;
(iv) antagonism of the BLT1 receptor may be determined by monitoring a compound's ability to block LTB₄-induced activation of BLT1 (intracellular calcium increase measured by a FLEX station).

Typically, an inhibitor of 5-LO, FLAP and/or LTA₄ hydrolase will have an: IC₅₀ for its target enzyme of 1 μM or less, preferably 100 nM or less. Similarly, an antagonist of the BLT1 receptor will have an IC₅₀ for BLT1 of 5 μM or less, preferably 250 nM or less. In all cases, the quoted IC₅₀ values are preferably those determined by way of an in vitro, cell-based assay (such as one of the assays mentioned above).

Specific inhibitors of 5-LO that may be mentioned include the following.
(1) Zileuton (synonyms: A-64077, ABT 077, Zyflo®), described in, for example, EP 0 279 263, U.S. Pat. No. 4,873,259, Int. J. Immunopharmacol. 14, 505 (1992), Br. J. Cancer 74, 683 (1996) and Am. J. Resp. Critical Care Med. 157, Part 2, 1187 (1998).

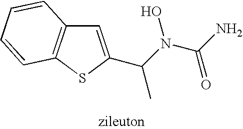

(2) A-63162, described in, for example, Anticancer Res. 14, 1951(1994).

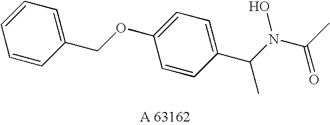

(3) A-72694.

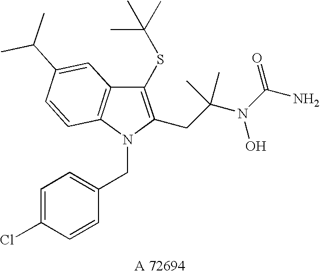

(4) A-78773, described in, for example, Curr. Opin. Invest. Drugs 2, 69 (1993).

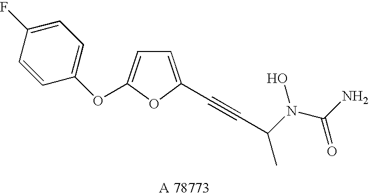

(5) A-79175 (the R-enantiomer of A 78773), described in, for example, Carcinogenesis 19, 1393 (1998) and J. Med. Chem. 40, 1955 (1997).

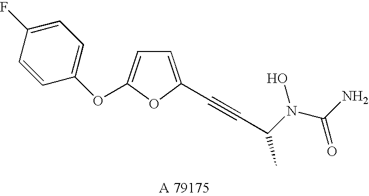

(6) A-80263.

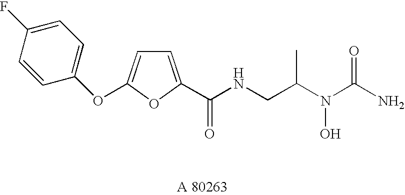

(7) A-81834.

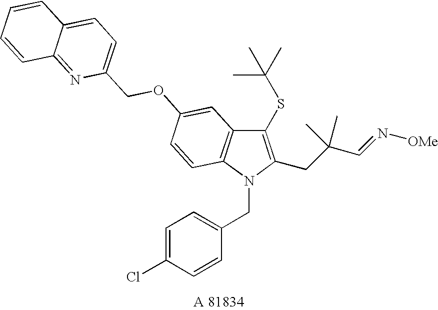

(8) A-93178

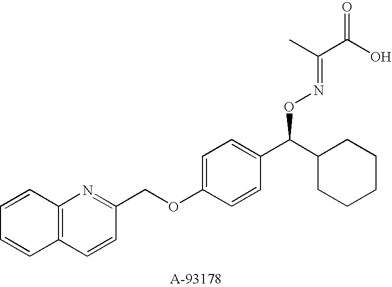

(9) A-121798, described in, for example, 211th Am. Chem. Soc. Meeting. 211: abstr. 246, 24 Mar. 1996.
(10) Atreleuton (synonyms ABT-761 and A-85761), described in, for example, Exp. Opin. Therap. Patents 5 127 (1995).

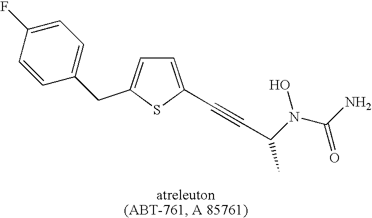

(11) MLN-977 (synonyms LPD-977 and CMI-977), described in, for example, Curr. Opin. Anti-Inflamm. & Immunomod. Invest. Drugs 1, 468 (1999). This, as well as similar compounds are described in U.S. Pat. No. 5,703,093.

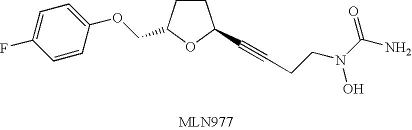

(12) CMI-947, described in, for example, 215th Am. Chem. Soc. Meeting. 215: abstr. MEDI 004, 29 Mar. 1998. This, as well as similar compounds are described in U.S. Pat. No. 5,792,776.

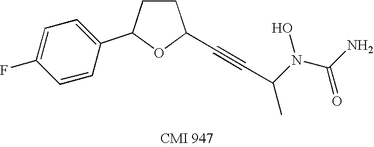

(13) CMI-568, described in, for example, 211th Am. Chem. Soc. Meeting. 211: abstr. 205, 24 Mar. 1996.

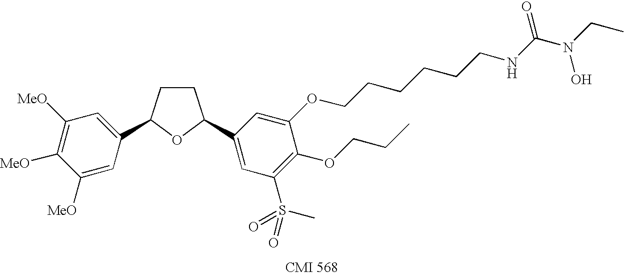

(14) LDP 392 (synonym CMI 392), described in, for example, Pharmacol. Res. 44, 213 (2001).

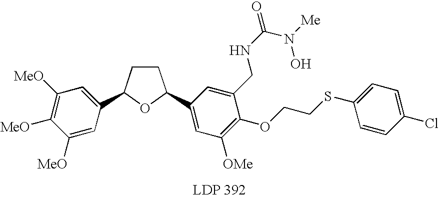

(15) Linetastine (synonyms: linazolast, TMK 688, YM 257), described in, for example, Int. J. Immunopharmcol. 22, 123 (2000).

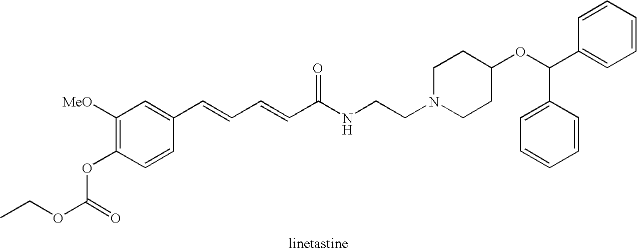

(16) Lonapalene (synonym: RS 43179), described in, for example, Pharm. Res. 9, 1145 (1992).

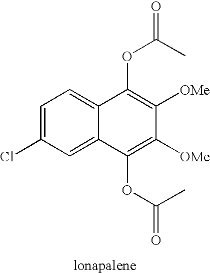

(17) LY-221068, described in, for example, Ann. N.Y. Acad. Sci. (Immunosuppressive and Antiinflammatory Drugs) 696, 415 (1993).

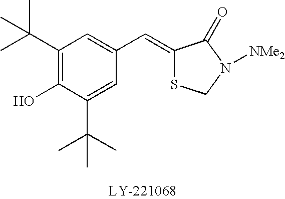

(18) LY 269415, described in, for example, Agents and Actions 42, 67 (1994).

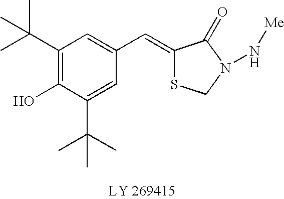

(19) 5-LO inhibitors with histamine H₁ receptor antagonist activity described in, for example, Bioorg. Med. Chem. Lett. 14, 2265 (2004), such as the following compound.

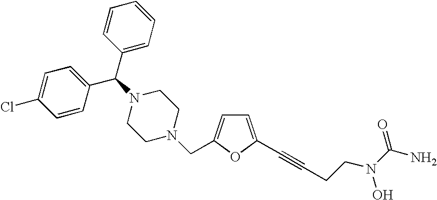

(20) BF-389

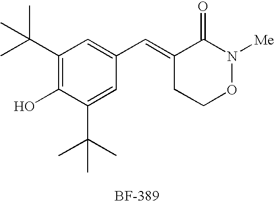

(21) BIL 226 and BIL 357, described in, for example, J. Pharmacol. Exp. Ther. 265, 483 (1993).

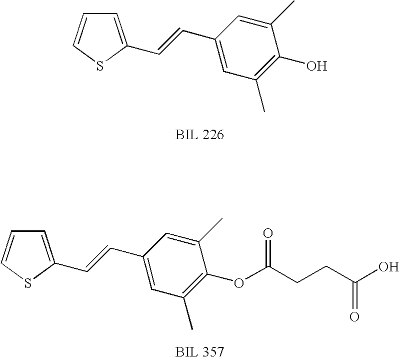

(22) BU 4601A, BU 4601B and BU 4601C, described in, for example, J. Antibiotics 46, 705 (1993).

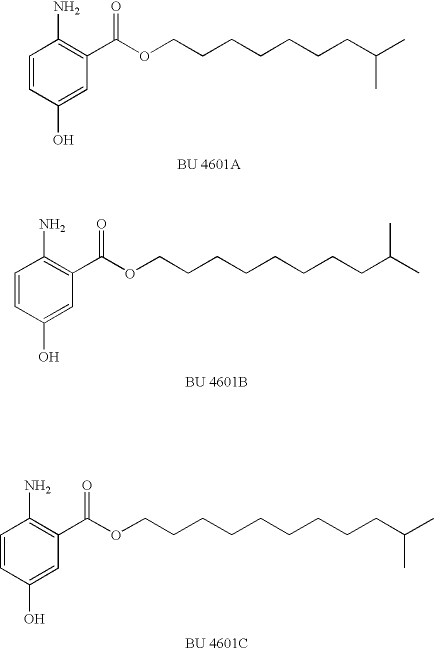

(23) BW 755C, described in, for example, J. Pharm. Exp. Therap. 277, 17 (1996).

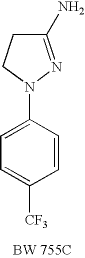

(24) BW-A4C, described in, for example, Eur. J. Biochem. 267, 3633 (2000).

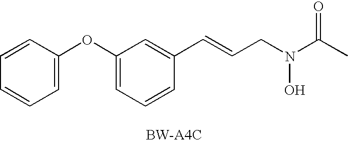

(25) BWB 70C, described in, for example, Br. J. Pharmacol. 108 (Suppl), 186P (1993).

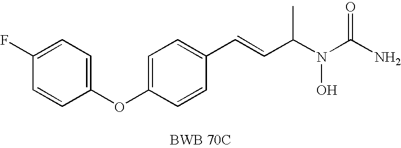

(26) CBS 1108.

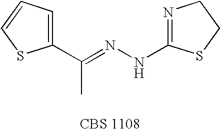

(27) CGS 26529, described in, for example, Inflamm. Res. 44 (Suppl. 2) 147 (1995).

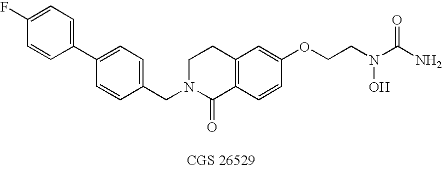

(28) CGS 25667, CGS 25997 and CGS 25998, described in, for example, J. Med. Chem. 38, 68 (1995).

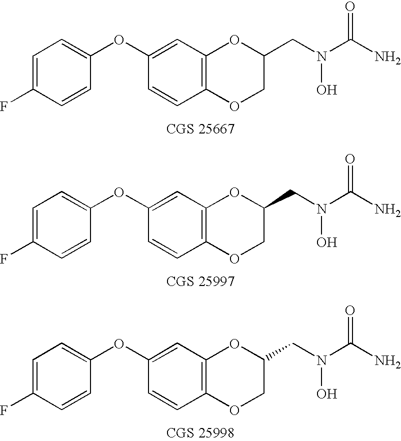

(29) CGS-23885, described in, for example, J. Med. Chem. 36, 3580 (1993).

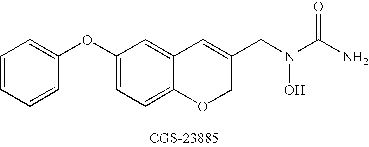

(30) CI-986

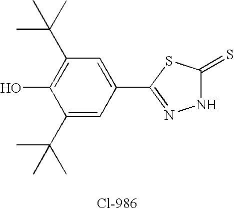

(31) CT 3 (synonyms: ajumelic acid, DMH-11C, HU 239), described in, for example, J. Med. Chem. 35, 3153 (1992).

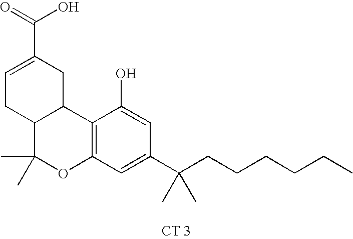

(32) CV 6504, described in, for example, Ann. Oncol. 11, 1165 (2000).

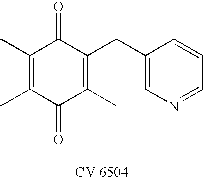

(33) Darbufelone (synonyms: CI-1004, PD 136095-0073) and analogues thereof, described in, for example, Arthritis and Rheumatism 42 (Suppl.) 404 (1999), ibid 42 (Suppl.) 81 (plus poster) (1999) and J. Med. Chem. 37, 322 (1994).

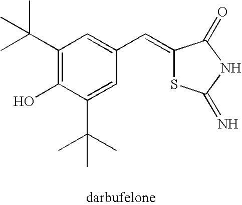

(34) Docebenone (synonym AA861) and analogues thereof, described in, for example, Int. Arch. Allergy and Immunol. 100, 178 (1993) and Biochim. Biophys. Acta 713, 470 (1982).

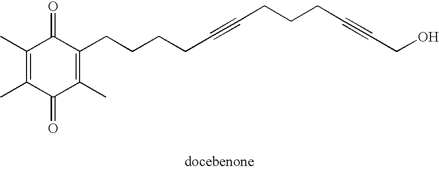

(35) DuP 654, described in, for example, J. Med. Chem. 33, 360 (1990).

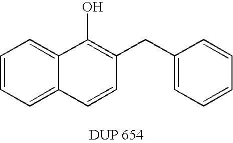

(36) XA 547, described in, for example, BTG International Inc. Company Communication 15 Oct. 1999, and Bioorg. Med. Chem. 3, 1255 (1995).

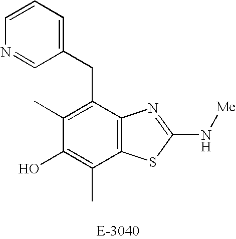

(38) E 6080, described in, for example, Res. Commun. Mol. Pathol. Pharmacol. 86, 75 (1994).

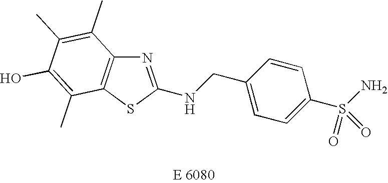

(39) E 6700.

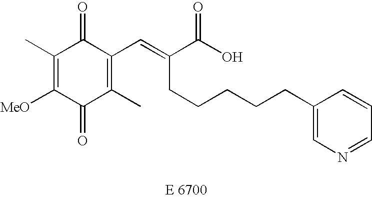

(40) Epocarbazolin A, a compound isolated from Streptomyces anulatus T688-8 and described in, for example, J. Antibiotics 46, 25 (1993).

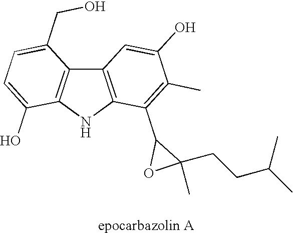

(41) ER 34122, described in, for example, Inflamm. Res. 47, 375 (1998).

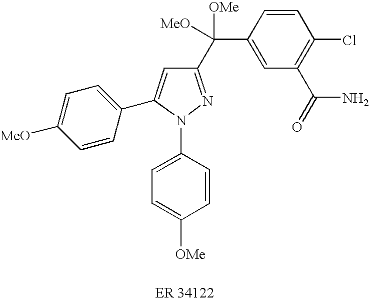

(42) ETH 615, described in, for example, Exp. Dermatol., 2, 165 (1993).

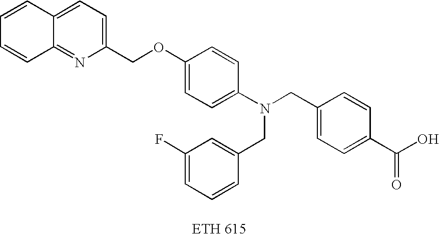

(43) F 1322, described in, for example, XV International Congress of Allergology and Clinical Immunology (Suppl 2) 325 (1994).

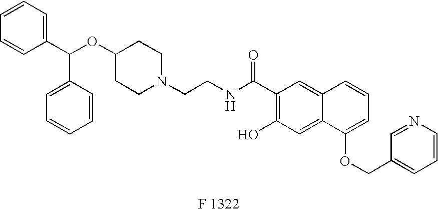

(44) Flezalastine (synonyms: D 18024, IDB 18024), described in, for example, Allergy (Suppl.) 47, 47 (1992).

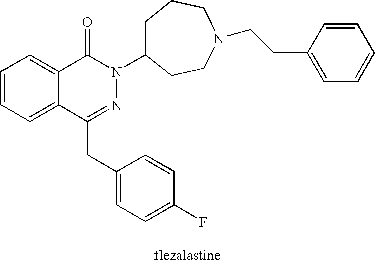

(45) Azelastine, described in, for example, Int. Arch. Allergy and Applied Immunol. 90, 285 (1989).

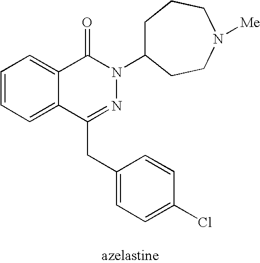

(46) FPL 62064, described in, for example, Agents and Actions 30, 432 (1990).

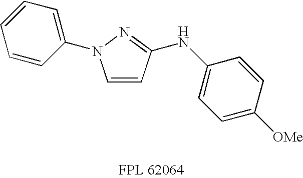

(47) FR 110302, described in, for example, Am. Rev. Resp. Dis. 145, A614 (1992).

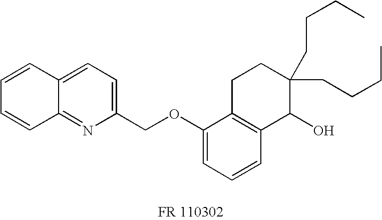

(48) HP 977 and P 10294, described in, for example, J. Med. Chem. 39, 246 (1996).

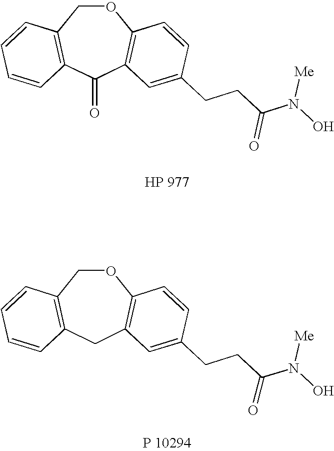

(49) P-8977

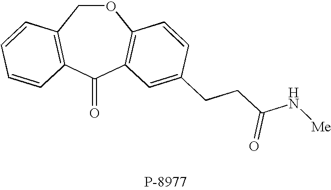

(50) HX-0835, described in, for example, Rinsho Iyaku. 11, 1577 & 1587 (1995).

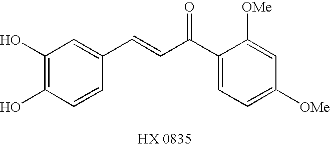

(51) HX-0836, described in, for example, J. Med. Chem. 36 3904 (1993).

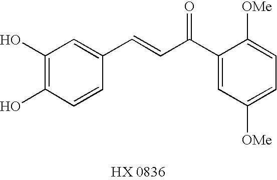

(52) The following compound, described in Bioorg. Med. Chem. Lett. 6, 93 (1996).

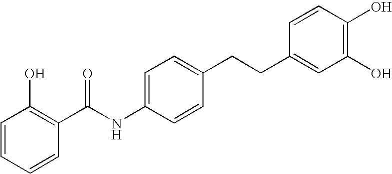

(53) Icodulinium (synonyms: CBS 113A, icoduline), described in, for example, Arzneimittel-Forschung (Drug Research) 39, 1242 & 1246 (1989).

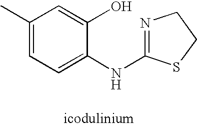

(54) KC-11404, described in, for example, Eur. Resp. J. 7 (Suppl. 18), 48 (1994).

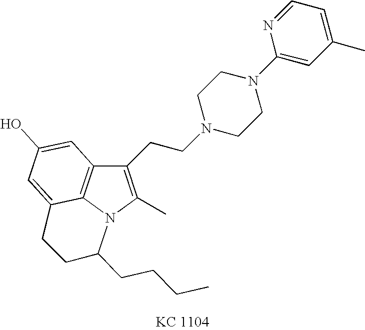

(55) KC-11425

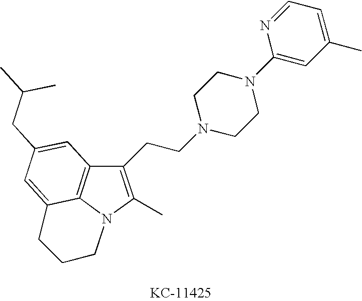

(56) KME 4.

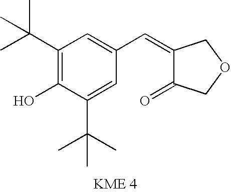

(57) L 651392, described in, for example, Adv. Prostaglandin, Thromboxane and Leukotriene Res. 17, 554 (1987).

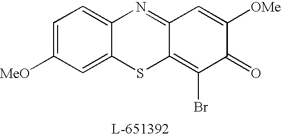

(58) L 651896.

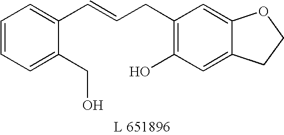

(59) L 652343.

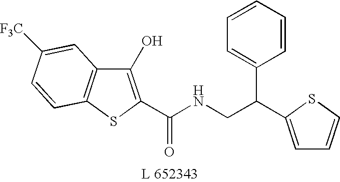

(60) L 653150.

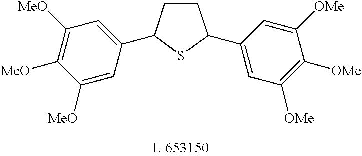

(61) L-656224, described in, for example, J. Gastroenterol. Hepatol. 11, 922 (1996).

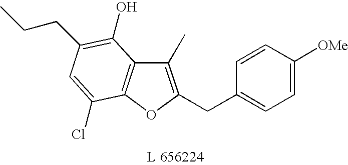

(62) L-702539, described in, for example, J. Med. Chem. 37, 512 (1994).

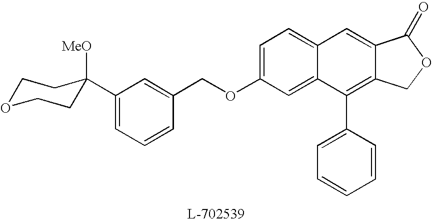

(63) L-670630.

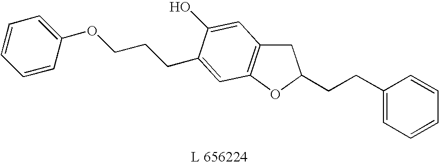

(64) L-691816, described in, for example, Curr. Opin. Invest. Drugs 2, 683 (1993).

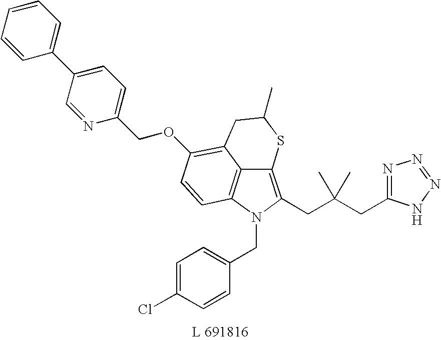

(65) L 699333, described in, for example, J. Med. Chem. 38, 4538 (1995).

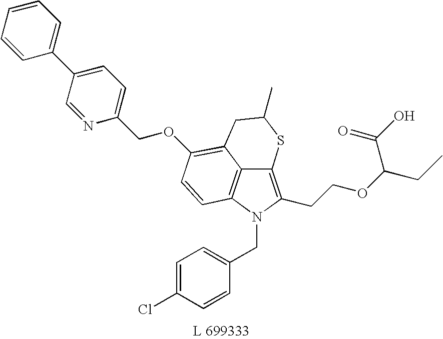

(66) L 739010.

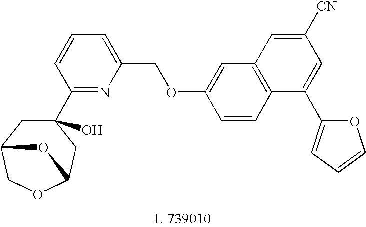

(67) Lagunamycin, described in, for example, J. Antibiotics 46, 900 (1993)

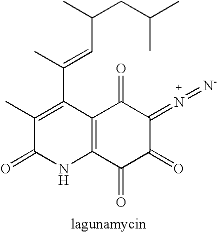

(68) Licofelone (synonym: ML 3000), described in, for example, Eur. J Pharm. 453, 131 (2002) and J. Med. Chem. 37, 1894 (1994).

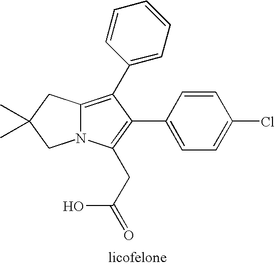

(69) PD 145246.

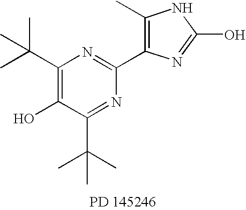

(70) R 840 (synonym: S 26431).

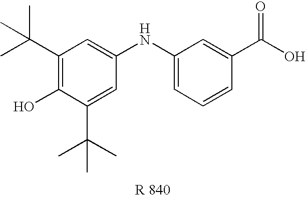

(71) R 68151, described in, for example, Arch. Dermatol. 128, 993 (1992).

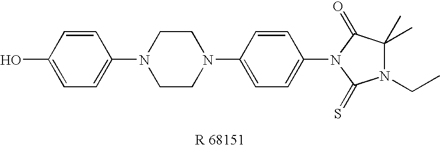

(72) R 85355, described in, for example, Skins Pharmacol. 9, 307 (1996).

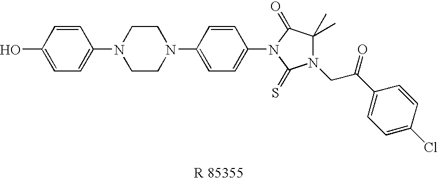

(73) REV 5901 (synonyms: PF 5901, Revlon 5901, RG 5901), described in, for example, J. Allergy Clin. Immunol. 91, 214 (1993).

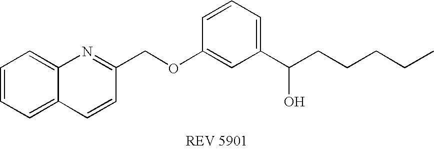

(74) RWJ 63556, described in, for example, 214th Am. Chem. Soc. Nat. Meeting. abstr. MEDI 091 (1997).

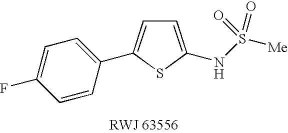

(75) S 19812, described in, for example, Mediators of Inflammation 8 (Suppl. 1), 134 & 135 (1999).

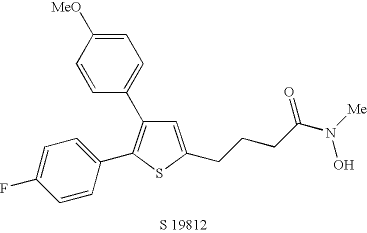

(76) SC 45662, described in, for example, J. Allergy and Clin. Immunol. 89, 208 (1992)

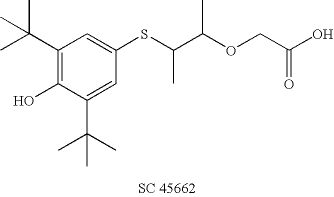

(77) SC-41661A

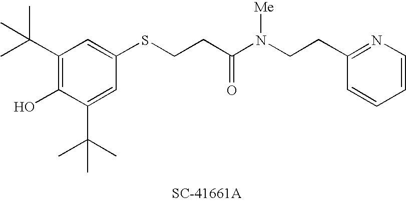

(78) SCH 40120.

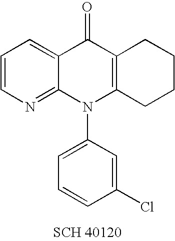

(79) SKF-86002

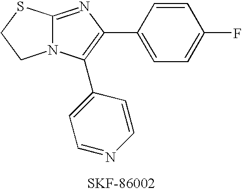

(80) SKF 104351 and SKF 105809.

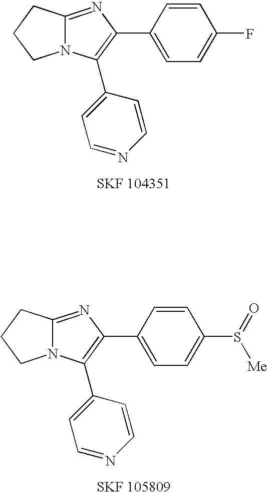

(81) SKF-107649, described in, for example, J. Med. Chem. 39, 5035 (1996).

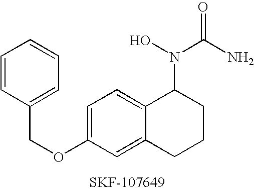

(82) T0757 and T0799), described in, for example, Jap. J. Pharmacol. 66, 363 (1994)

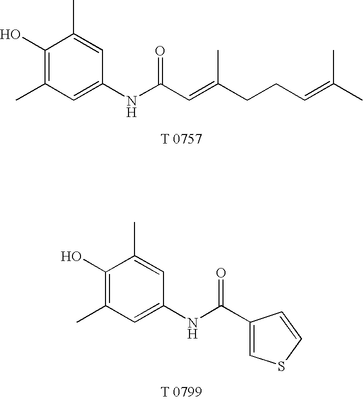

(83) TA 270, described in, for example, Naunyn-Schmiedeberg's Arch. Pharmacol. 358 (Suppl. 2) 737 (1998).

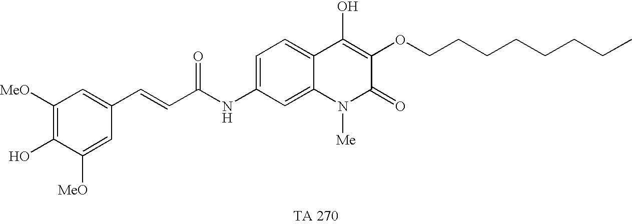

(84) Tagorizine (synonym: AL 3264), described in, for example, Jap. J Pharmacol. 65, 19 (1994) and ibid. 64 (Suppl. 1), 312 (1994)

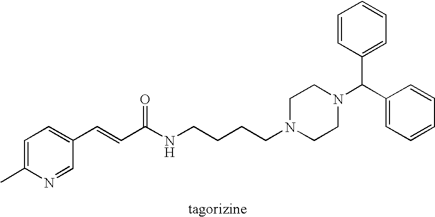

(85) Tepoxalin (synonyms: ORF 20485, RWJ 20485), described in, for example, J. Pharmacol. Exp. Therap. 271, 1399 (1994).

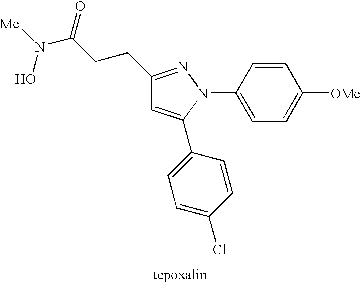

(86) UPA 780, described in, for example, Inflamm. Res. 44 (Suppl. 3), 273 (1995).

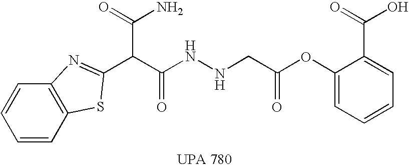

(87) VUFB 19363.

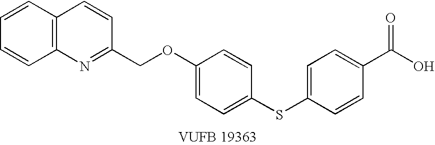

(88) VZ 564, described in, for example, Arzneimittel-Forschoung (Drug Research) 25, 155 (1995).

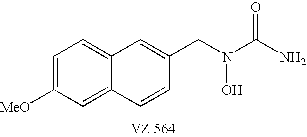

(89) The following compound, described in J. Med. Chem. 40, 819 (1997).

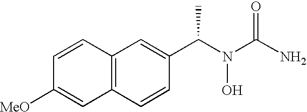

(90) WAY 120739.

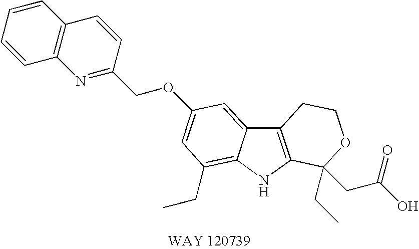

(91) WAY 121520, described in, for example, Agents and Action 39 (Spec. issue C1) C30 (1993) and Exp. Opin. Invest. Drugs 6, 279 (1997).

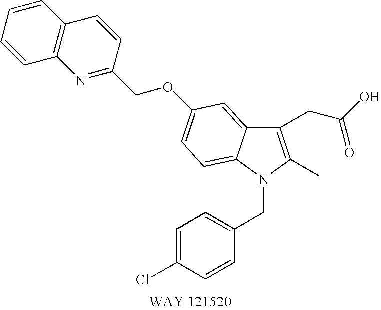

(92) WAY-126299A, described in, for example, Inflamm. Res. 44 (Suppl. 2), 170 (1995).

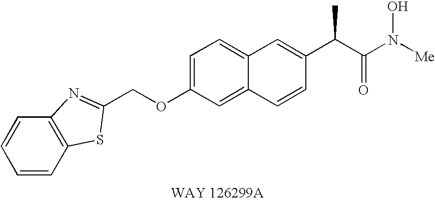

(93) WAY-125007, described in, for example, WO 04/004773

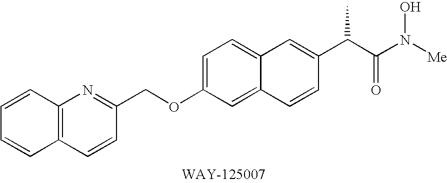

(94) WHIP 97, described in, for example, 216th Am. Chem. Soc. Nat. Meeting abstr. MEDI 363 (1998).

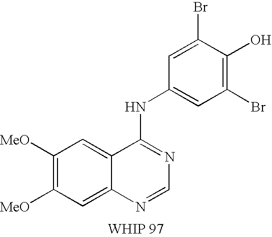

(95) WY 28342, described in, for example, J. Med. Chem. 38, 1473 (1995).

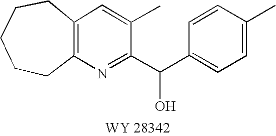

(96) WY 50295 (the S-enantiomer of WY 49232), described in, for example, Eur. J. Pharmacol. 236, 217 (1993).

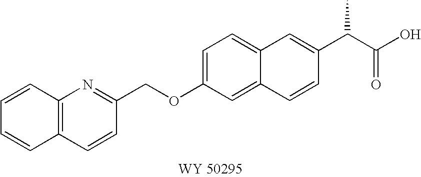

(97) ZD 2138 (synonym: ICI D 2138), described in, for example, Asthma 95: Theory to Treatment 15 (1995) and Trends in Pharm. Sci. 13, 323 (1992).

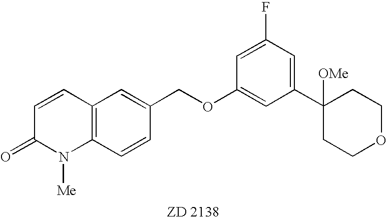

(98) ZM 230487 (synonym: ICI 230487), described in, for example, Inpharma 660, 9 (1994).

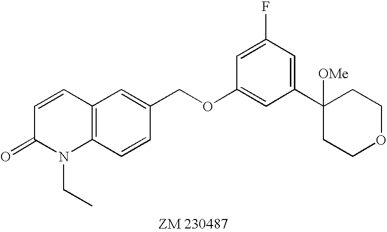

(99) ZD 4007 and ZD 4407, described in, for example, EP 0 623 614.

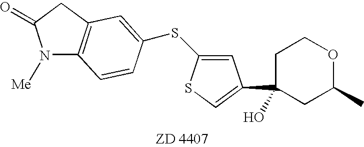

(100) ZD 7717, described in, for example, EP 0 462 813.

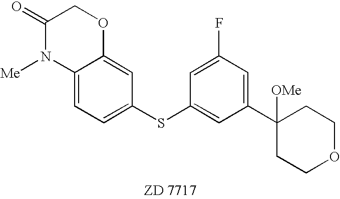

(101) ZM-216800

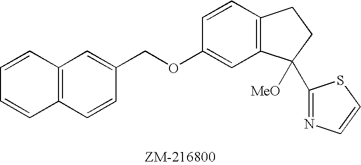

(102) CJ-12,918, and analogues thereof, described in, for example, Bioorg. Med. Chem. 11, 3879 (2003).

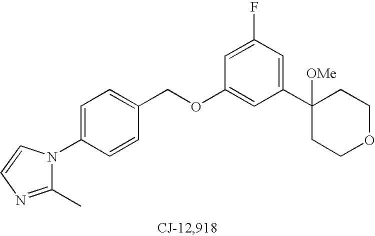

(103) Compounds described as mixed 5-LO/COX-2 inhibitors in Biorg. Med. Chem. Lett. 12, 779 (2002), such as the following compound.

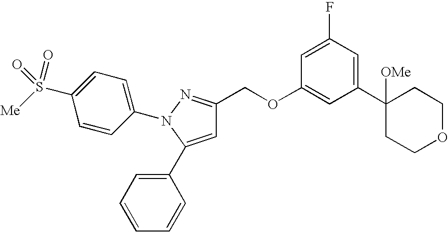

(104) AKBA (acetyl-11-keto-β-boswellic acid), described in, for example, Br. J. Pharmacol. 117, 615 (1996) and Eur. J. Biochem. 256, 364 (1998).

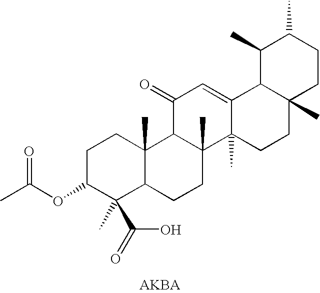

(105) Compounds described as dual 5-LO and COX inhibitors in Eur. J. Med. Chem. 22, 147 (1997) and Arzeimittel-Forschung (Drug Research) 35, 1260 (1985), such as 2-acetylthiophene-2-thiazolylhydrazone (CBS-1108) and N-phenylbenzamidrazone.

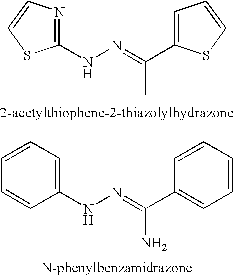

(106) Boswellin (an extract from Boswellia serrata), described in, for example, Fifth Chemical Congress of North America, Abstract 01/1351 (1997) and ibid. Abstract 01/1350 (1997).
(107) 2,4,6-triiodophenol, described as a 5-LO inhibitor in, for example, U.S. Pat. No. 5,985,937.
(108) Nicaraven, described in, for example, Curr. Opin. Invest. Drugs 4, 83 (2003).

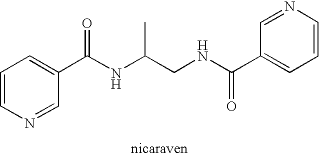

(109) Tenidap, described in, for example, EP 0 156 603, U.S. Pat. No. 4,556,672, Arthritis Rheum. 31, Suppl. S52 (1988) and P. Katz et al., ibid. S52.

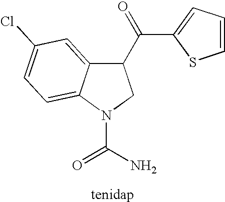

(110) Cyclic hydrazides described as 5-LO inhibitors in J. Med. Chem. 39, 3938 (1996), such as phenidone, 1-phenyl-2H-tetrahydropyridazin-3-one, and 1-phenylperhydro-1,2,4-tetrahydropyridazin-3-one.

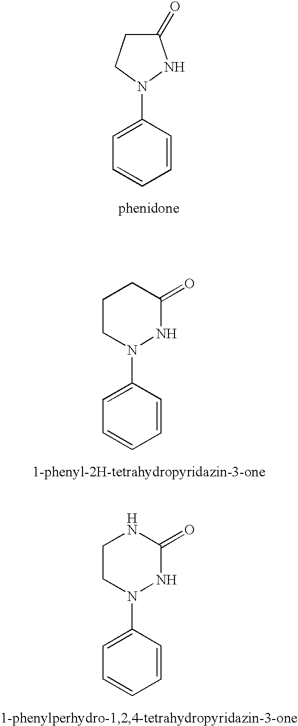

(111) ICI 207968, described in, for example, J. Med. Chem. 34, 1028 (1991).

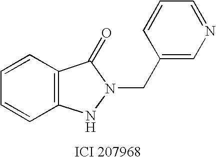

(112) ICI 211965, and other (methoxyalkyl)thiazoles, described in, for example, J. Med. Chem. 34, 2176 (1991).

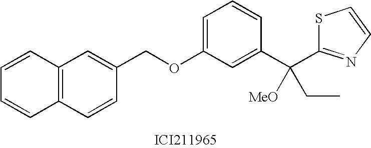

(113) 2,3-Dihydro-5-benzofuranols described in J. Med. Chem. 32, 1006 (1989), such as the following compound.

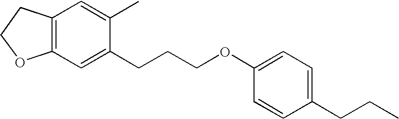

(114) 2,6-Di-tert-butylphenol derivatives described in Bioorg. Med. Chem. 11, 4207 (2003), such as tebufelone, R-830, and S2474.

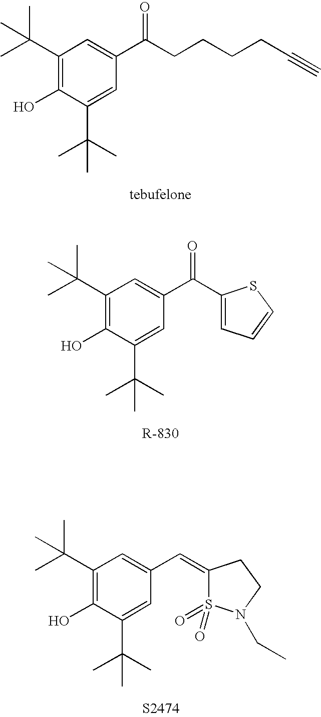

(115) 7-tert-Butyl-2,3-dihydro-3,3-dimethylbenzofurans described as 5-LO/COX-2 inhibitors in J. Med. Chem. 41, 1112 (1998), such as PGV-20229.

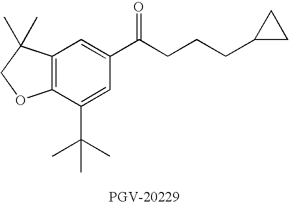

(116) Compounds described as dual 5-LO/COX inhibitors in Eur. J. Med. Chem. 35, 1897 (2003), such as the following compound.

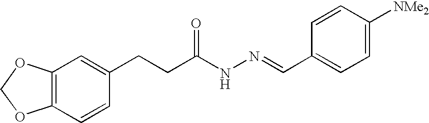

(117) Helenalin, a sesquiterpene lactone that can be isolated from several plant species of the Asteraceae family, described in, for example Biochem. Pharm. 62, 903 (2001).
(118) AS-35, (9-[(4-acetyl-3-hydroxy-2-n-propylphenoxy)methyl]-3-(1H-tetrazol-5-yl)-4H-pyrido[1,2-a]pyrimidin-4-one), described in, for example, Int. J. Immunopharmacol. 22, 483 (2000).

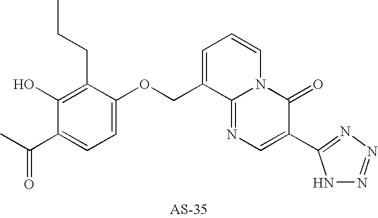

(119) Magnolol, described in, for example, Planta Medica 65, 222 (1999).

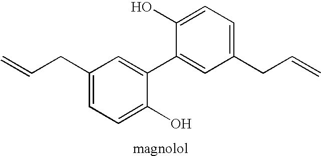

(120) Honokiol, extracted from Chinese herbal medicine, and described in, for example, Arch. Allergy and Immunol. 110, 278 (1996).

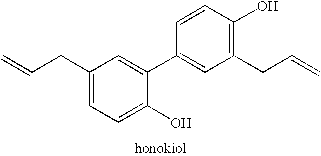

(121) Chrysarobin.

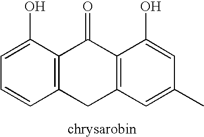

(122) E-3040.

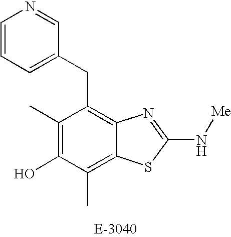

(123) Flobufen, described in, for example, Chirality 16, 1 (2004).

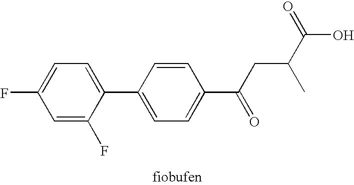

(124) YPE-01, described in, for example, Eur. J. Pharmacol. 404, 375 (2000).

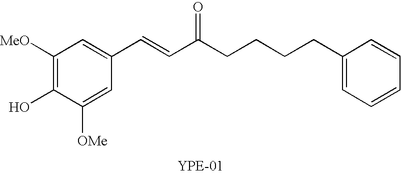

(125) BW-A137C

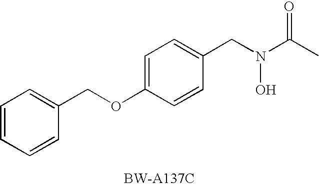

(126) LY-233569

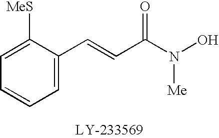

(127) PD-138387

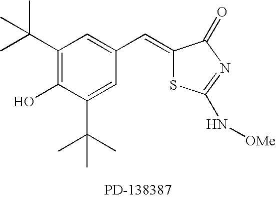

(128) SB-210661

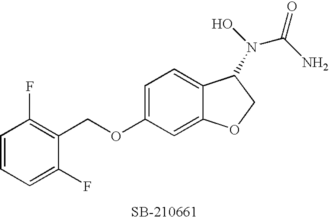

(129) DuP-983

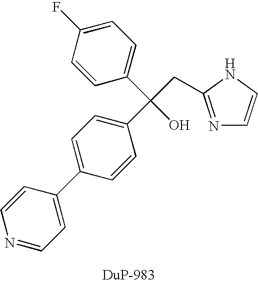

(130) BTS-71321

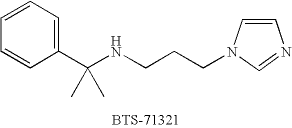

(131) Piripost, described in, for example, Toxicon. 24, 614 (1986).
(132) MK-866, described in, for example, Eur J. Pharmacol 205, 259 (1991).
(133) UCB 62045, described in, for example, Chest 123, 371S (2003).
(134) ONO-LP-049, described in, for example, J. Immunol. 140, 2361 (1988).
(135) 3323W, L-697198, L-7080780, FR-122788, CMI-206, FPL-64170 and PD-089244.

Other specific 5-LO inhibitors that may be mentioned include those described in the review articles Prog. Med. Chem., G. P. Ellis and D. K. Luscombe, Elsevier 29, 1 (1992) and J. Med. Chem. 14, 2501 (1992).

Specific inhibitors of FLAP that may be mentioned include the following.
(a) L-674,573, and related FLAP inhibitors (e.g. L-655,238), described in, for example, Mol. Pharmacol. 40, 22 (1991).

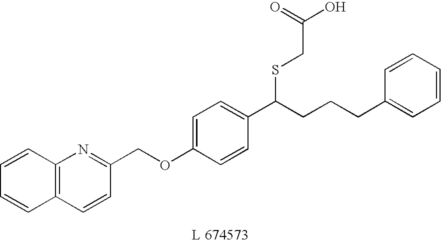

(b) L-674,636, described in, for example, J. Med. Chem. 38, 4538 (1995).

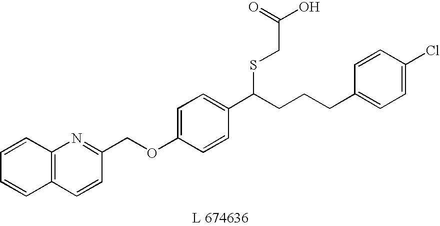

(c) L-689,037, and photoaffinity analogues [¹²⁵I]-669,083 and [¹²⁵I]-691,678, described in, for example, Mol. Pharmacol. 41, 267 (1992).

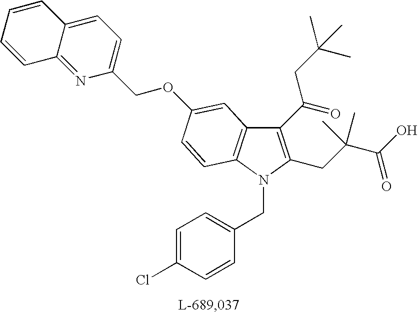

(d) L-705,302, described in, for example, J. Med. Chem. 38, 4538 (1995).

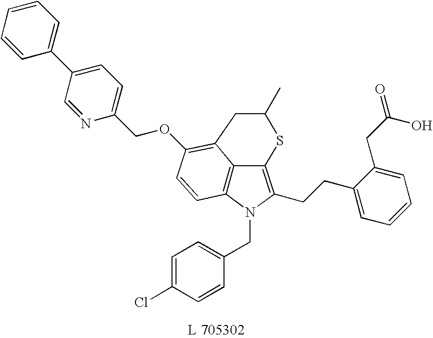

(e) MK-886 (synonyms: L663536, MK 0886), described in, for example, U.S. Pat. No. 5,081,138, Am. Rev. Resp. Dis. 147, 839 (1993), Eur. J. Pharmacol. 267, 275 (1994), The Search for Anti-Inflammatory Drug. 233 (1995) Eds.: V. J. Merluzzi and J. Adams, Boston, Birkhäuser.

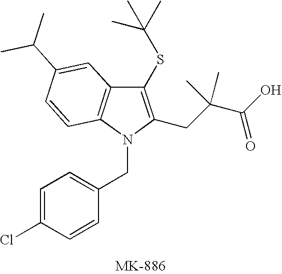

(f) Compounds structurally related to MK-886, described in, for example, WO 93/16069, U.S. Pat. No. 5,308,850 and WO 94/13293
(g) Quiflapon (synonyms: MK-591, L 686708), described in, for example, J. Physiol. Pharmacol. 70, 799 (1992) and J. Lipid Mediators 6, 239 (1993).

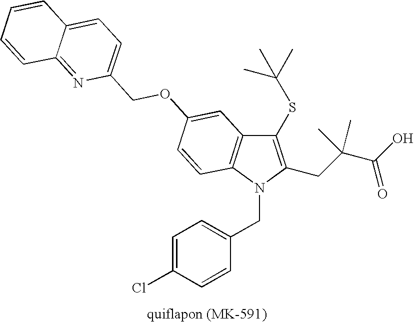

(h) BAY X 1005, described in, for example, Thorax 52, 342 (1997).

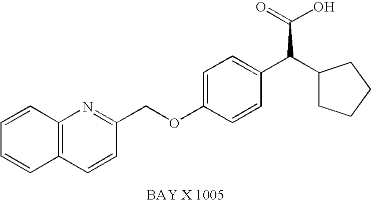

(i) BAY Y 105, described in, for example, Arthritis and Rheumatism 39, 515 (1996) and Drug & Market Devel. 7, 177 (1996).

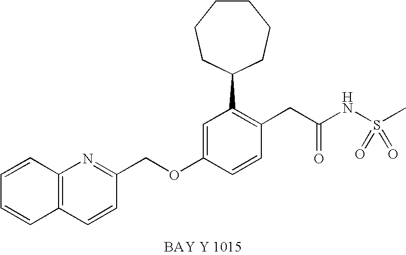

(j) VML 530 (synonym: ABT 080), described in, for example, Pharmacologist 39, 33 (1997).

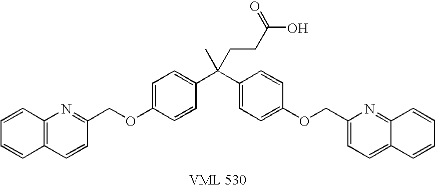

### Inhibitors of LTA₄ hydrolase that may be mentioned include the following.

(A) Compounds described as LTA hydrolase inhibitors in U.S. Pat. No. 5,455,271 and WO 94/00420, for example:

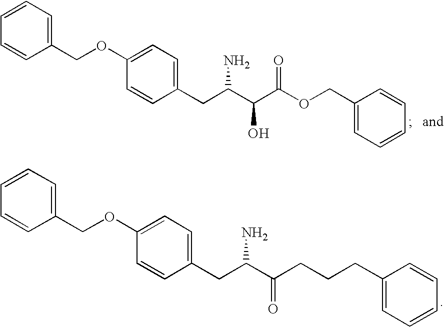

(B) Compounds described as LTA₄ hydrolase inhibitors in J. Med. Chem. 36, 211 (1993) and J. Am. Chem. Soc. 114, 6552 (1992), such as the following compound.

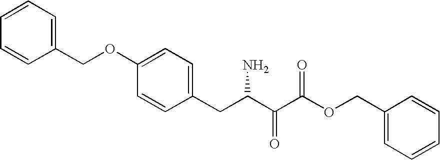

(C) Compounds identifiable by the method of claim 24 of WO 00/50577.
(D) Compounds described as LTA₄ hydrolase inhibitors in U.S. Pat. No. 6,506,876, such as SC-56938.

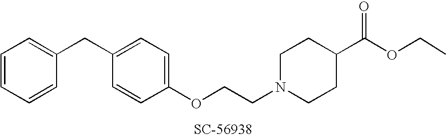

(E) Analogues of SC-56938, described in, for example, Bioorg Med. Chem. Lett. 12, 3383 (2002).
(F) Compounds described as LTA₄ hydrolase inhibitors in U.S. Pat. No. 5,719,306, for example:

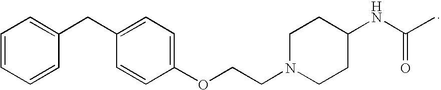

(G) Compounds described as LTA₄ hydrolase inhibitors in WO 96/11192, such as:

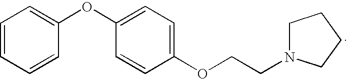

(H) Compounds described as LTA₄ hydrolase inhibitors in U.S. Pat. No. 6,265,433 and WO 98/40364, for example:

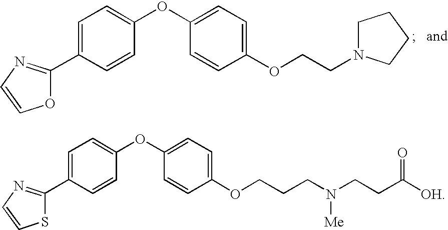

(I) Compounds described as LTA₄ hydrolase inhibitors in U.S. Pat. No. 6,506,876 and WO 96/10999, such as:

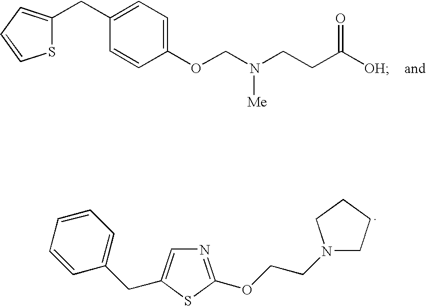

(J) Compounds described as LTA₄ hydrolase inhibitors in WO 98/40370, such as:

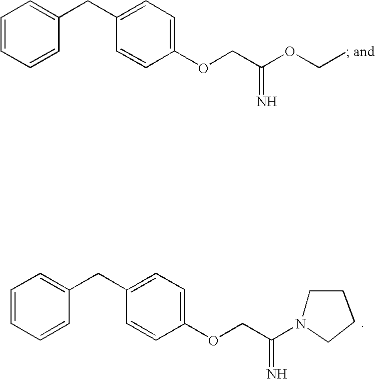

(K) Compounds described as LTA₄ hydrolase inhibitors in WO 98/40354.
(L) Compounds (3-oxiranylbenzoic acids) described as LTA₄ hydrolase inhibitors in EP 0 360 246, such as:

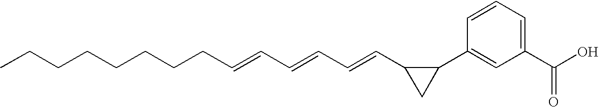

(M) 20,20,20-Trifluoroleukotriene B4 derivatives, described in, for example, JP 01211549 A2, such as the following compound.

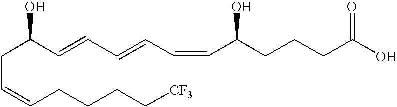

(N) Compounds described as LTA₄ hydrolase inhibitors in EP 1 165 491 and WO 00/059864, such as 2-amino-6-(4-benzylphenoxy)hexanoic acid:

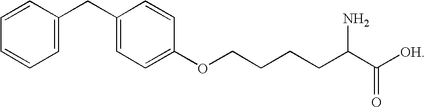

(0) Compounds described as LTA₄ hydrolase inhibitors in U.S. Pat. No. 6,436,973 and WO 00/017133, such as (2S,3R)-2-amino-3-(benzyloxy)butane-1-thiol:

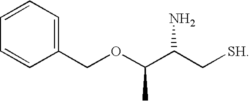

(P) Compounds described as LTA₄ hydrolase inhibitors in Bioorg Med. Chem. 3, 969 (1995), such as:

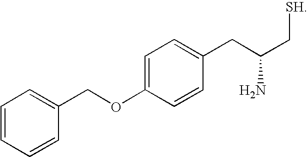

(Q) [4-(ω-Arlalkyl)phenyl]alkanoic acids described as LTA₄ hydrolase inhibitors in DE 4121849 A1, such as:

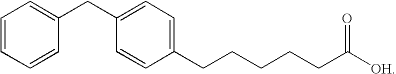

(R) Aralkylthienylakaoates described as LTA₄ hydrolase inhibitors in DE 4118173 A1, such as:

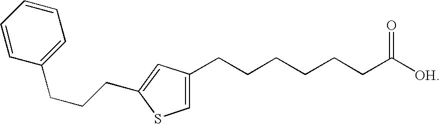

(S) ω-[(4-Arylalkyl)thien-2-yl]alkanoates described as LTA₄ hydrolase inhibitors. in DE 4118014 A1, such as:

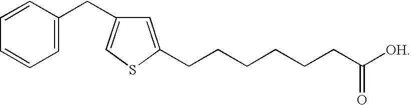

(T) Compounds described as LTA₄ hydrolase inhibitors in J. Med. Chem. 35, 3156 (1992), such as RP64966:

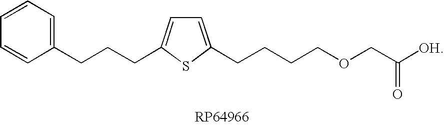

(U) Compounds structurally related to RP66153 and described in J. Med. Chem. 35, 3170 (1992).
(V) 2-Hydroxyphenyl-substituted isoxazoles described as LTA₄ hydrolase inhibitors in DE 4314966 A1, such as:

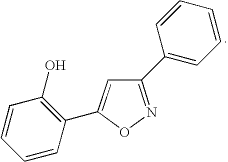

(W) Bestatin, described in, for example, J. Nat. Cancer Institute 95,1053 (2003).

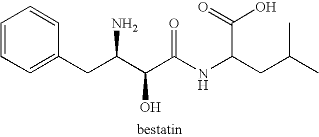

(X) SC-22716 (1-[2-(4-phenylphenoxy)ethyl]pyrrolidine), described in, for example, J. Med. Chem. 43, 721 (2000).

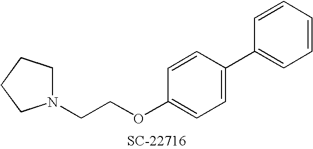

(Y) SC57461A, described in, for example, J. Med. Chem. 45, 3482 (2002) and Curr. Pharm. Design 7, 163 (2001).

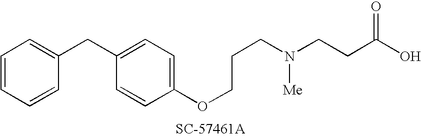

(Z) Imidazopyridines and purines described as LTA₄ hydrolase inhibitors in Bioorg. Med. Chem. Lett. 13, 1137 (2003).
(AA) Captopril, described in, for example, FASEB Journal 16, 1648 (2002).

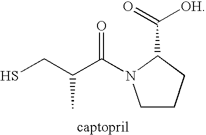

(AB) Hydroxamic acid derivatives described as LTA₄ hydrolase inhibitors in WO 99/40910, such as:

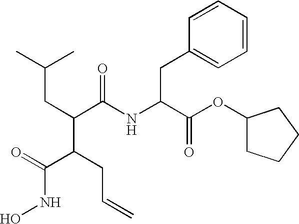

(AC) AB5366, described in, for example, JP 11049675 A2.
(AD) SA6541, described in, for example, WO 96/27585, Life Sci. 64, PL51-PL56 (1998) and Eur. J. Pharmacol. 346, 81 (1998).

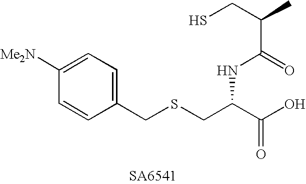

(AE) Compounds containing N-mercaptoacylprolines described as LTA₄ hydrolase inhibitors in JP 10265456 A2, such as:

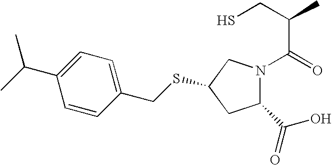

(AF) 14,15-Dehydroleukotriene A4, described in, for example, Biochem. J. 328,225 (1997).
(AG) 8(S)-amino-2(R)-methyl-7-oxononanoic acid, produced by Streptomyces diastaticus and described in, for example, J. Natural Products 59, 962 (1996).

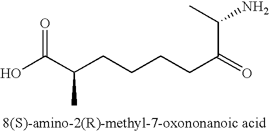

(AH) The hydroxamic acid-containing peptide kelatorphan, described in, for example, Bioorg. Med. Chem. Lett. 5, 2517 (1995).
(AI) Amino hydroxamic acids described as inhibitors of LTA₄ hydrolase in Bioorg. Med. Chem. 3, 1405 (1995), such as:

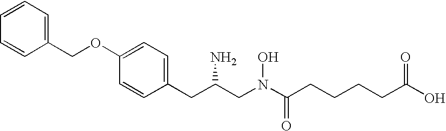

(AJ) α-Keto-β-amino esters and thioamines described as inhibitors of LTA₄ hydrolase in J. Pharmacol. Exp. Therap. 275, 31 (1995).
(AK) N-(phenylbutanoyl)leucines described as inhibitors of LTA4 hydrolase in JP 05310668 A2.

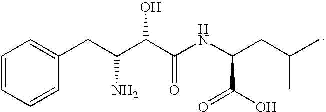

Other specific inhibitors of LTA₄ hydrolase that may be mentioned include those described in the review articles Curr. Pharm. Design 7, 163 (2001) and Curr. Med. Chem. 4, 67 (1997).

Antagonists of LTB₄ receptors (e.g. BLT1) that may be mentioned include the following.
(i) Compounds described as LTB₄ receptor antagonists in U.S. Pat. No. 6,291,530, such as (E)-[5-(2-diethylcarbamoyl-1-methylvinyl)-2-(2,6-difluoro-benzyloxy)phenoxy]acetic acid:

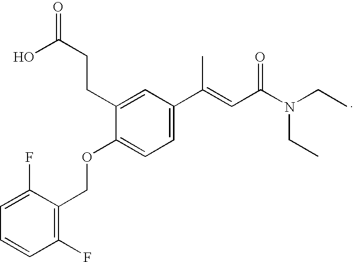

(ii) Compounds described as LTB₄ receptor antagonists in US 2002/0128315, such as 4-(4-phenylpiperidinylmethyl)benzoic acid 4-amidinophenyl ester and 4-(2-phenylimidazolylmethyl)benzoic acid 4-amidinophenyl ester:

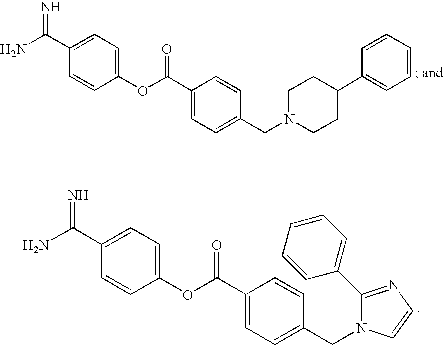

(iii) Compounds described as LTB₄ receptor antagonists in US 2004/0053962, such as 2-(2-propyl-3-(3-(2-ethyl-4-(4-fluorophenyl)-5-hydroxyphenoxy)propoxy)phenoxy)benzoic acid:

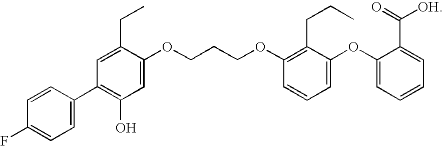

(iv) BIIL, described in, for example, J. Pharmacol. Exp. Therap. 297, 458 (2001) and WO 02/055065.

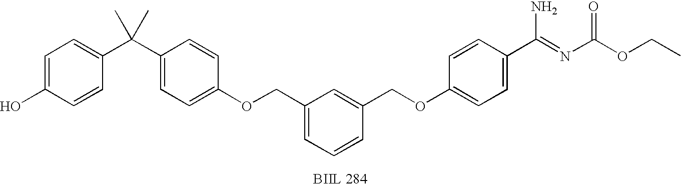

(v) CP 105696 and CP 195543, described in, for example, J. Pharmacol. Exp. Therap. 285, 946 (1998).

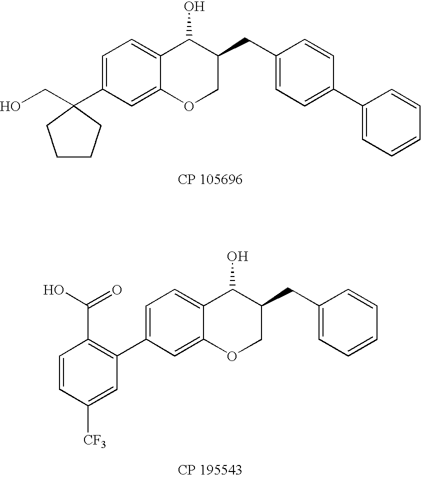

(vi) LY 210073

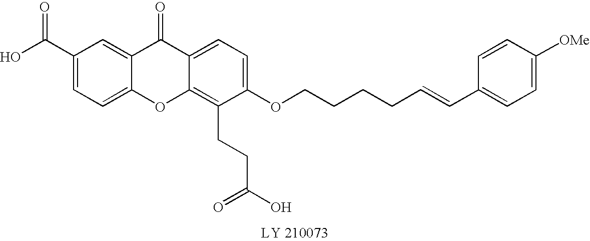

(vii) LY 223982 (synonyms: CGS 23131, SKF 107324).

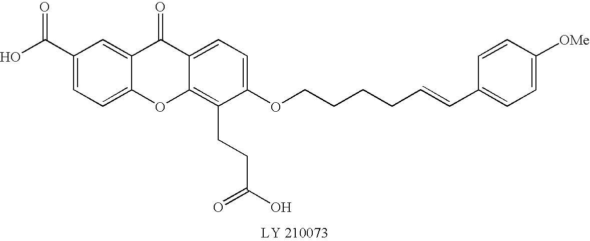

(viii) LY 255283 (synonyms: CGS 23356, LY 177455), described in, for example, Eur. J. Pharmacol 223, 57 (1992).

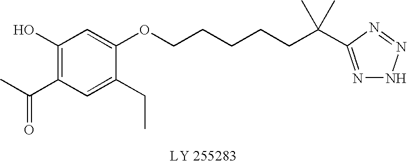

(ix) LY 292728.

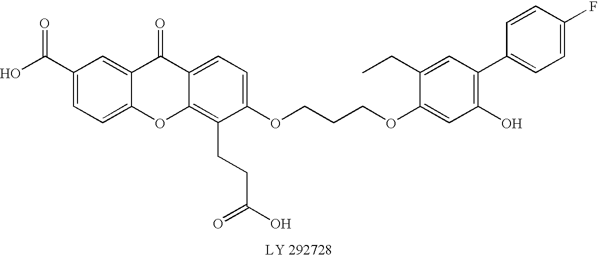

(x) LY 293111 (synonym: VML 295), described in, for example, Drugs of the Future 21, 610 (1996), Clin. Cancer Res. 8, 3232 (2002) and WO 01/085166.

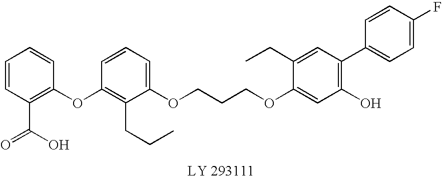

(xi) LTB 019.
(xii) Moxilubant (synonym: CGS 25019C), described in, for example, Exp. Opin. Therap. Patents 5, 127 (1995).

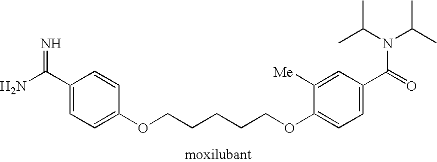

(xiii) Olopatidine (synonyms: allelock, ALO 4943A, KW 4679, Patanol®), described in, for example, Drugs of the Future 18, 794 (1993).

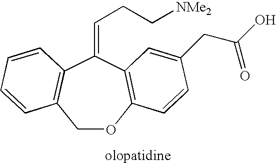

(xiv) ONO 4057 (synonym: LB 457), described in, for example, Gastroenterology 110 (Suppl.), 110 (1996).

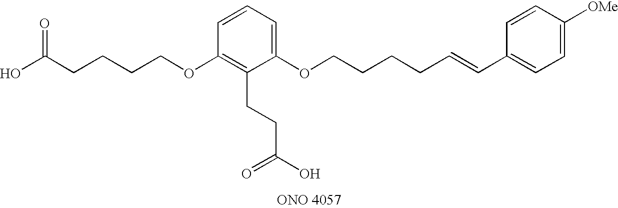

(xv) Ontazolast (synonym: BIRM 270), described in, for example, J. Pharm. Exp. Therap. 271, 1418 (1994).

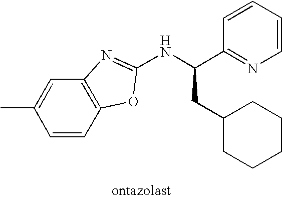

(xvi) PF 10042, described in, for example, Eur. J. Pharmacol. Environmental Toxicology and Pharmacology Section 293, 369 (1995).

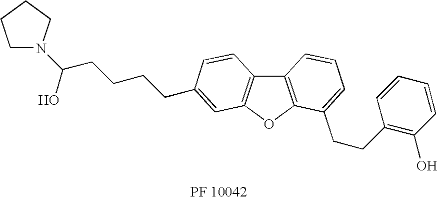

(xvii) RG 14893, described in, for example, Pharmacologist 34, 205 (1992).

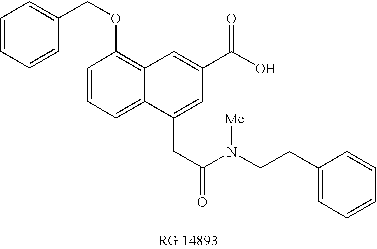

(xviii) RO 254094, described in, for example, ISSX Proceedings 6, 232 (1994).

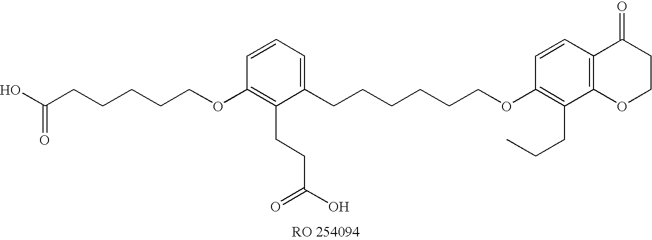

(xix) RP 66153.

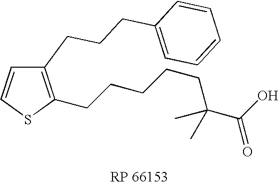

(xx) RP 66364.

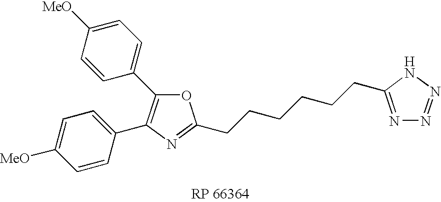

(xxi) RP 69698.

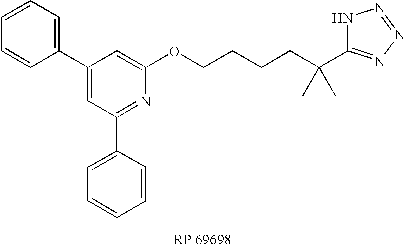

(xxii) SB 201146, described in, for example, Thorax 53, 137 (1998).

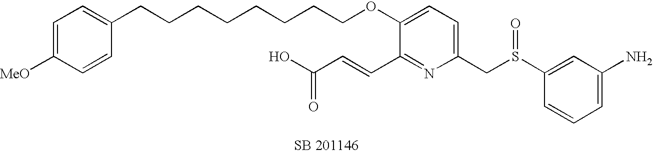

(xxiii) SB 201993, described in, for example, J. Med. Chem. 36, 2703 (1993).

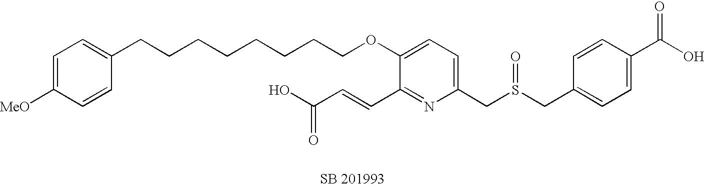

(xxiv) SC 41930, described in, for example, J. Pharmacol. Exp. Therap. 269, 917 (1994).

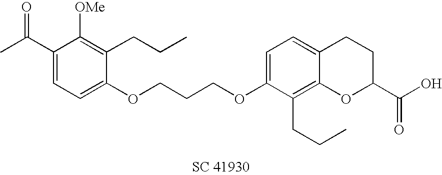

(xxv) SC 50605.

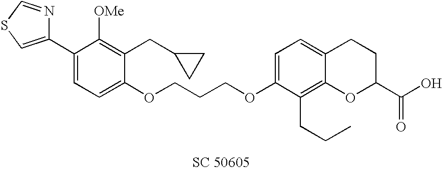

(xxvi) SC 51146.

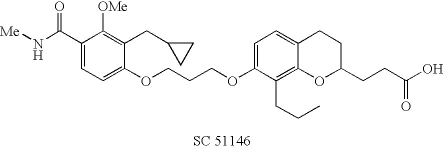

(xxvii) SC 53228, described in, for example, Inflammation Res. 44 (Suppl. 2), 143 (1995).

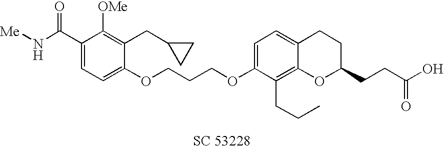

(xxviii) Ticolubant (synonym: SB 209247), described in, for example, Adv. Prostaglandin Thromboxane and Leukotriene Res. 23, 275 (1995).

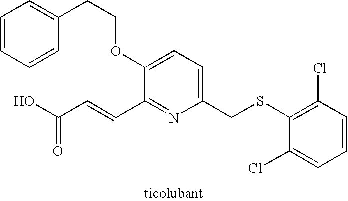

(xxix) U 75302 (synonyms: U 75485, U 77692, U 78489), described in, for example, Adv. Prostaglandin Thromboxane and Leukotriene Res. 23, 275 (1995).

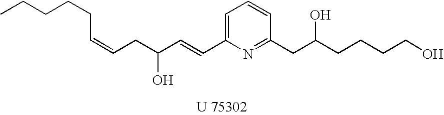

(xxx) VM 301 (synonyms: OAS 1000, pseudopterosin A methyl ether), described in, for example, Inflammation Res. 44, (Suppl. 3) 268 (1995).
(xxxi) ZD 158252, described in, for example, Inpharma 1094, 9 (1997).
(xxxii) ZK 158252, described in, for example, Inpharma 1094, 9 (1997).

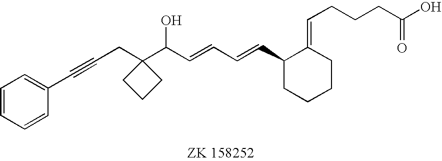

(xxxiii) U-75509, described in, for example, Am. J. Physiol. Heart Circ. Physiol. 2004, Mar 11 [Epub ahead of print].
(xxxiv) CP-105,696, described in, for example, Br. J. Pharmacol. 139, 388 (2003).
(xxxv) LY293111, described in, for example, Clin. Cancer Res. 8, 3232 (2002).

The compounds listed or referred to above are commercially available, may be prepared by techniques known to those skilled in the art from materials that are commercially available, and/or may be prepared by methods that are identifiable via the documents mentioned above (i.e. detailed in those documents or in documents identified therein). The disclosures of the documents mentioned above that describe specific compounds that inhibit the synthesis and/or function of LTB₄ are hereby incorporated by reference.

Patients (e.g. human patients) in need of treatment by the method of the present invention include those determined by standard diagnostic methods as suffering from B-CLL, B-PLL or B-cell lymphoma (e.g. determination of whether the patient is experiencing fever, anemia, perspiration and/or fatigue—see also, for example: Epidemiol. Rev. 20, 187 (1998); Blood 87, 4990 (1996); J. Clin. Oncol. 17, 3835 (1999); Cancer 48, 198 (1981); and Blood 46, 219 (1975)).

The term “cancer chemotherapeutic agent having a different mechanism of action”, when used herein includes any compound, other than an inhibitor of the biosynthesis and/or function of LTB₄, that can be used to treat cancer.

The term thus includes the following agents.
(a) Alkylating agents including:
   (i) nitrogen mustards such as mechlorethamine (HN₂), cyclophosphamide, ifosfamide, melphalan (L-sarcolysin) and chlorambucil;
   (ii) ethylenimines and methylmelamines such as hexamethylmelamine, thiotepa;
   (iii) alkyl sulfonates and thiosulfonates such as busulfan, methyl methanesulfonate (MMS) and methyl methanethiosulfonate;
   (iv) nitrosoureas and nitrosoguanidines such as carmustine (BCNU), lomustine (CCNU), semustine (methyl-CCNU), streptozocin (streptozotocin) and N-methyl-N′-nitro-N-nitrosoguanidine (MNNG); and
   (v) triazenes such as dacarbazine (DTIC; dimethyltriazenoimidazole-carboxamide).
(b) Antimetabolites including:
   (i) folic acid analogues such as methotrexate (amethopterin);
   (ii) pyrimidine analogues such as fluorouracil (5-fluorouracil; 5-FU), floxuridine (fluorodeoxyuridine; FUdR) and cytarabine (cytosine arabinoside); and
   (iii) purine analogues and related inhibitors such as mercaptopurine (6-mercaptopurine; 6-MP), thioguanine (6-thioguanine; TG) and pentostatin (2′-deoxycoformycin).
(c) Natural Products including:
   (i) vinca alkaloids such as vinblastine (VLB) and vincristine;
   (ii) epipodophyllotoxins such as etoposide and teniposide;
   (iii) antibiotics such as dactinomycin (actinomycin A, C, D or F), daunorubicin (daunomycin; rubidomycin), doxorubicin, bleomycin, plicamycin (mithramycin) and mitomycin (mitomycin A, B or C);
   (iv) enzymes such as L-asparaginase; and
   (v) biological response modifiers such as interferon alphenomes.
(d) Miscellaneous agents including:
   (i) platinum coordination complexes such as cisplatin (cis-DDP) and carboplatin;
   (ii) anthracenedione such as mitoxantrone and anthracycline;
   (iii) hydroxyurea;
   (iv) methyl hydrazine derivatives such as procarbazine (N-methylhydrazine, MIH);
   (v) adrenocortical suppressants such as mitotane (o,p′-DDD) and aminoglutethimide;
   (vi) taxol and analogues/derivatives;
   (vii) hormone agonists/antagonists such as flutamide and tamoxifen;
   (vii) photoactivatable compounds (e.g. psoralens);
   (ix) DNA topoisomerase inhibitors (e.g. m-amsacrine and camptothecin);
   (x) anti-angiogenesis agents (e.g. SU6668, SU5416, combretastatin A4, angiostatin and endostatin); and
   (xi) immunotherapeutic agents (e.g. radiolabelled antibodies such as Bexxar™ and Theragyn™ (Pemtunomab™)).

In relation to the third and fourth aspects of the invention, the term “is administered” includes administration of the other cancer chemotherapeutic agent (i.e. the agent having a different mechanism of action) prior to, during and/or following treatment of the patient with the inhibitor of the biosynthesis and/or function of LTB₄. Administration of the other cancer chemotherapeutic agent preferably takes place within the period of 48 hours before and 48 hours after (e.g. within the period of 24 hours before and 24 hours after) treatment with this medicament. It is particularly preferred that administration takes place within the period of 12 hours before and 12 hours after (e.g. within the period of 6 hours before and 6 hours after) treatment, such as i the period of 3 hours before and 3 hours after treatment or within the period of 2 to 5 hours before treatment. Administration of multiple doses of the other cancer chemotherapeutic agent and/or the inhibitor of the biosynthesis and/or function of LTB₄ are also contemplated.

In such cases, the relative time scales mentioned above relate to the time separation between administration of neighbouring doses of the other cancer chemotherapeutic agent and the inhibitor of the biosynthesis and/or function of LTB₄.

The term “pharmaceutically acceptable derivative” includes references to salts (e.g. pharmaceutically-acceptable non-toxic organic or inorganic acid addition salts) and solvates.

The method described herein may have the advantage that, in treating B-CLL, B-PLL or B-cell lymphoma, it may be more convenient for the physician and/or patient than, be more efficacious than, be less toxic than, have a broader range of activity than, be more potent than, produce fewer side effects than, or that it may have other useful pharmacological properties over, similar methods (treatments) known in the prior art.

### BRIEF DESCRIPTION OF THE FIGURES

FIG. 1 depicts the level of biosynthesis of LTB₄ by B-CLL cells under various conditions. B-CLL cells (10×10⁶) were:

incubated for five minutes at 37° C. with calcium ionophore A23187 (final concentration 1 μM);

incubated for five minutes at 37° C. with arachidonic acid (AA) (final concentration 40 μM);

incubated for five minutes at 37° C. with A23187 (1 μM) plus arachidonic acid (40 μM);

sonicated and subsequently incubated for five minutes at 37° C. with ATP (1 mM), calcium chloride (2 mM) and arachidonic acid (40 μM); or

pre-incubated (intact cells) with diamide (100 μM) for two minutes, followed by stimulation with A23187 (1 μM) and arachidonic acid (40 μM).

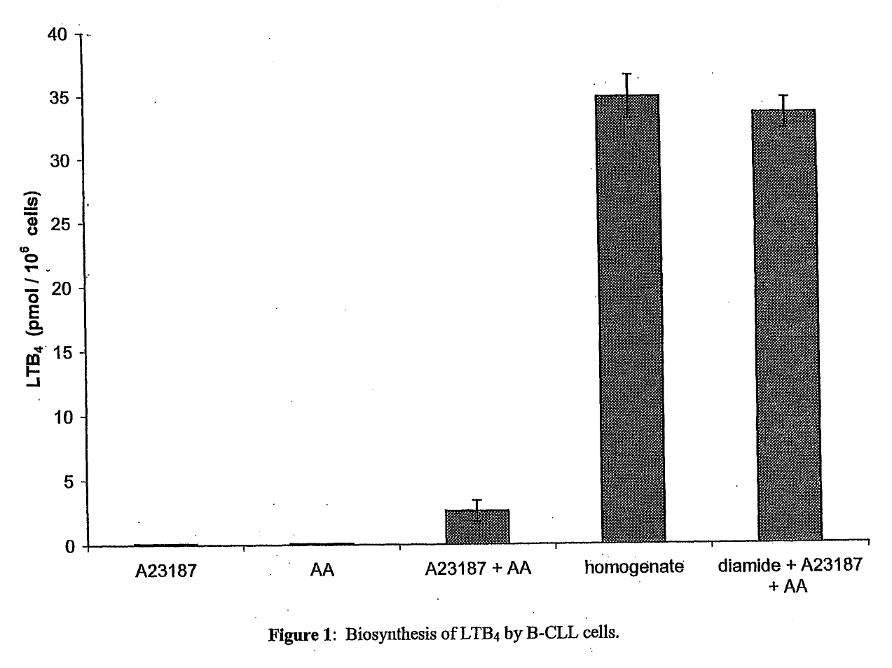

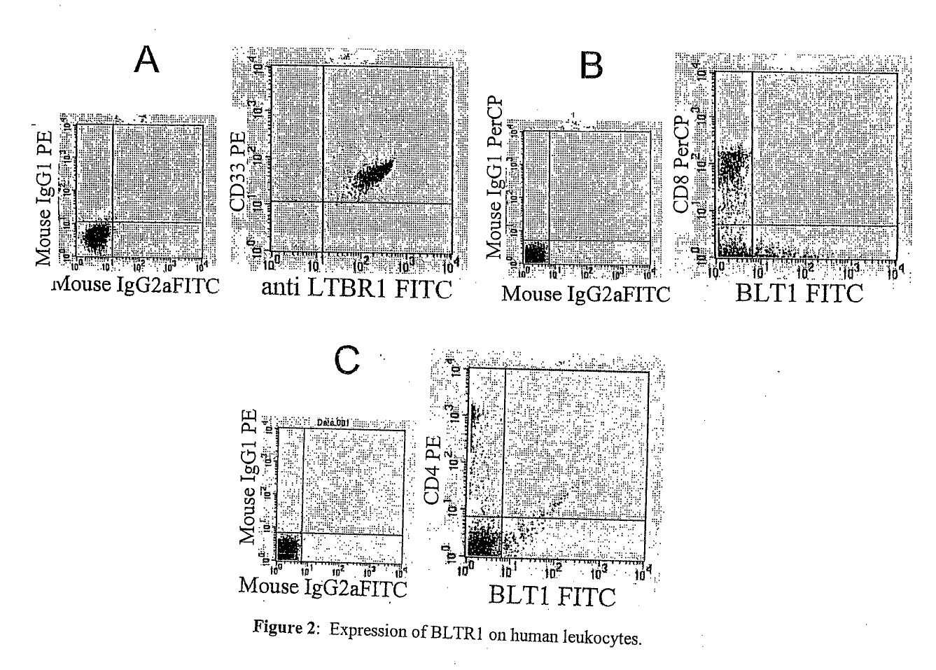

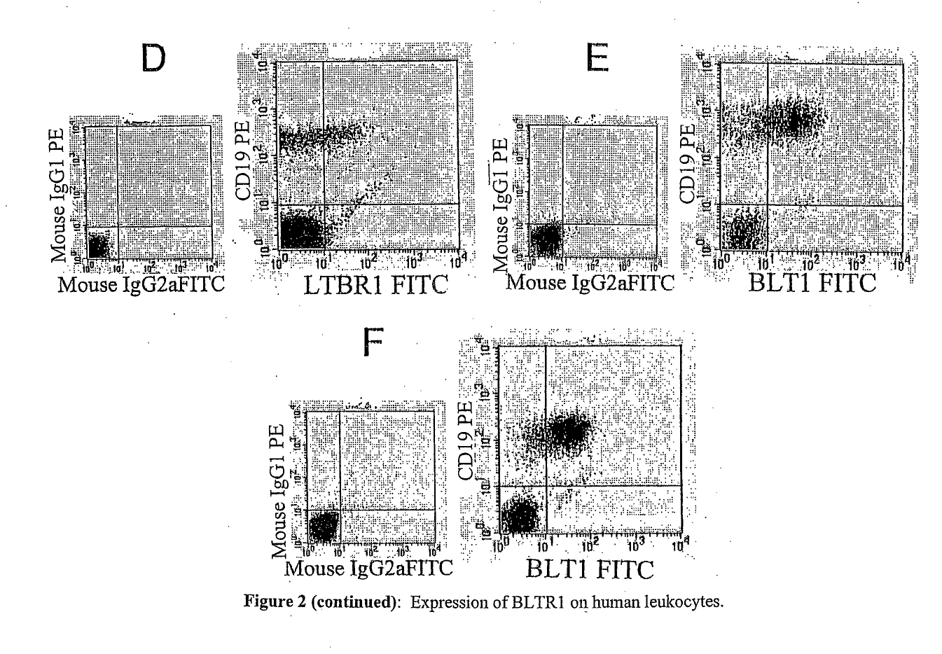

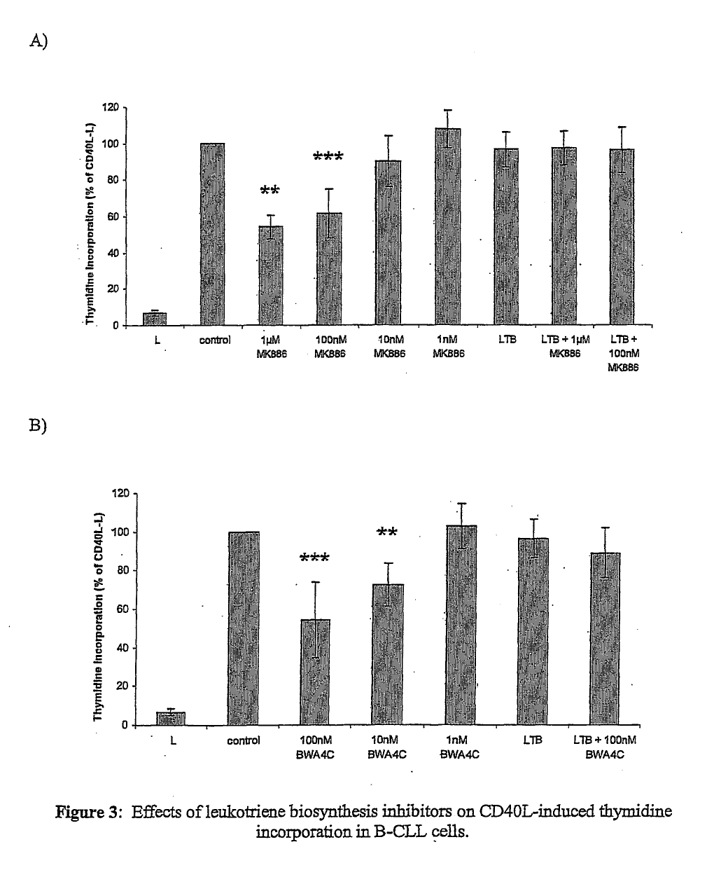

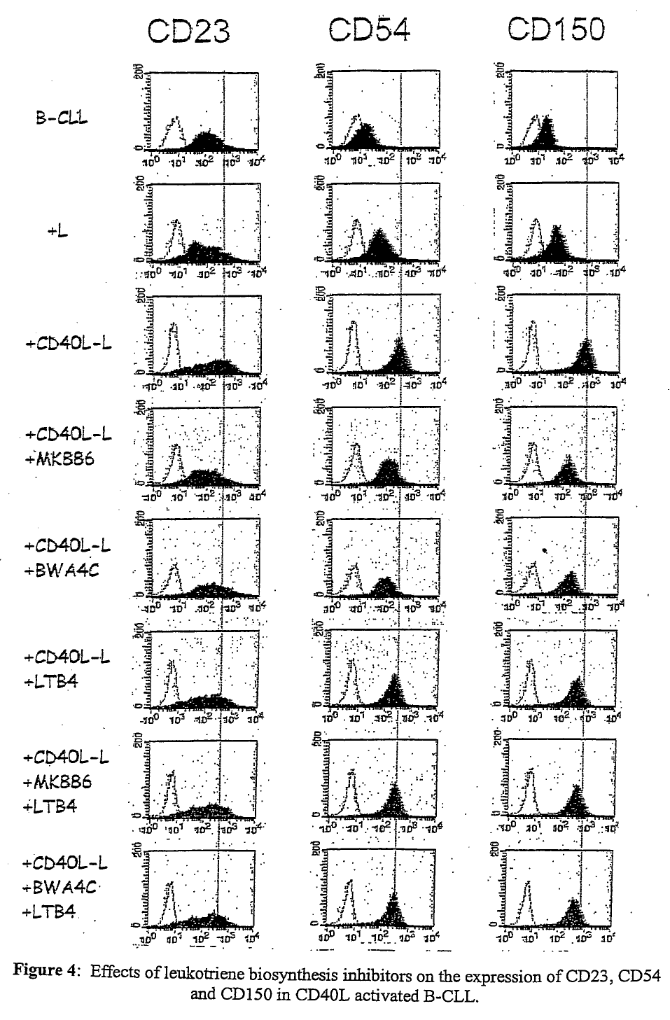

Values given in FIG. 1 are mean±S.D. of six independent experiments.

FIG. 2 depicts the expression of BLTR1 on human leukocytes. The expression BLTR1 was analysed in various leukocytes by FACS. The specific leukocytes were:

A) PMNL;

B) peripheral CD8⁺T-cells;

C) peripheral CD4⁺T-cells;

D) normal peripheral B-cells;

E) B-CLL cells; and

F) B-PLL cells.

In all of the graphs depicted in FIG. 2, the large panel shows expression of BLTR1 and the cell specific antigen, whereas the small panel shows results with negative control antibodies. The figure depicts one typical experiment out of six except for B-PLL (two experiments).

FIG. 3 depicts the effects of leukotriene biosynthesis inhibitors on CD40L-induced thymidine incorporation in B-CLL cells. B-CLL cells (2×10⁵) were co-cultured with either irradiated L cells alone (L), irradiated CD40L-L cells or irradiated CD40L-L cells plus indicated inhibitor for 96 hr. When inhibitors were used, B-CLL cells were pre-treated with the inhibitor for 30 min prior co-culturing with L cells or CD40L-L cells. The inhibitors used were:

A) MK886 (10⁻⁶ to 10⁻⁹ M (10⁻⁶ M was only used in three experiments); or

B) BWA4C (10⁻⁷ to 10⁻⁹ M),

with or without LTB₄ (10⁻⁷ M) for 96 hrs in triplicates. The control result reported in the Figure represents B-CLL cells co-cultured with irradiated CD40L-L cells alone. ³H-thymidine (1 μCi) was present for the final eight hours. Activation of B-CLL cells with CD40L-L treatment led to between 3580 and 15369 cpm (³H-thymidine) incorporation (control) in different experiments. This was set as 100% in each experiment. The results show the mean±S.D from eight separate experiments (B-CLL cells from two patients were analyzed two times). Student's t-test was used to calculate statistics i.e. control vs. control plus indicated compound(s) (** P<0.01, *** P<0.001).

FIG. 4 depicts the effects of leukotriene biosynthesis inhibitors on the expression of CD23, CD54 and CD150 in CD40L activated B-CLL. Purified B-CLL cells were co-cultured with either L cells or CD40L-L cells in the absence or presence of MK886 (10⁻⁷ M), BWA4C (10⁻⁷ M), and/or LTB₄ (10⁻⁷ M) for 96 hrs. When inhibitors were used, B-CLL cells were pre-treated with the inhibitor for 30 min prior to co-culturing with L cells or CD40L-L cells. B-CLL cells were collected and analysed by FACS with antibodies against CD23, CD54 or CD150. The figure depicts one typical experiment out of six. In order to more clearly demonstrate the different degree of expression of indicated antigen in the various samples, the inserted dotted line represents the expression of the indicated antigen in B-CLL cells stimulated with CD40L-L alone.

### Biological Tests

### Materials and Methods

### Reagents and Cell Lines:

The calcium ionophore A23187 was purchased from Calbiochem-Behring (La Jolla, Calif., U.S.A.). HPLC solvents were obtained from Rathburn chemicals (Walkerburn, U.K.) and the synthetic standards of LTB₄ and prostaglandin (PG) B. were from Biomol (Plymouth meeting, Pa., U.S.A.). BWA4C was a kind gift from Lawrie G Garland, Wellcome Research Laboratories, UK and MK-886 from Jilly F. Evans, Merck Frosst Centre for Therapeutic Research, CA. Azodicarboxylic acid bis(dimethylamide) (diamide) was purchased from Sigma (Stockholm, SE). Mouse fibroblastic L cells transfected with the human CD40L (CD40L⁺L cells) were used for activation and untransfected L cells (CD40L⁻) as control (see J. Exp. Med. 182, 1265 (1995)).

### Isolation of Cells:

B-cells were isolated from patients suffering with B-CLL or B-prolymphocytic leukemia (B-PLL) who had not received chemotherapy within during the previous six weeks (see Table 1 below).

TABLE 1
Clinical data on patients with B-CLL.

| Patient | | | | Survival | Sample |
|---|---|---|---|---|---|
| No. | Sex | Age | Rai | (Months) | (Months from diagnosis) |
| 1 | F | 51 | 0 | 234+ | 232 |
| 2 | F | 67 | 0 | 114+ | 48 |
| 3 | M | 72 | I | 62+ | 57 |
| 4 | M | 66 | III | 91+ | 88 |
| 5 | M | 63 | I | 101+ | 90 |
| 6 | F | 68 | 0 | 37+ | 35 |

(Patient data and Rai stadium at diagnosis. Survival is measured as months from diagnosis (+ means that patients are still alive). Patients 3 and 6 have never received treatment. The other patients have received several courses of therapy with one to six different regiments.)

Peripheral blood samples were obtained after informed consent and with local ethics committee approval. Blood samples were Ficoll-Isopaque purified and washed twice in phosphate buffered saline (PBS). After that, cells were either frozen in PBS with 50% human AB serum and 10% dimethylsulfoxide or analyzed fresh. Frozen cell samples were thawed and washed in ice cold fetal calf serum and subsequently in PBS before analysis. Cells from two patients were used twice, both freshly isolated cells and after freezing with similar results. However, similar results were obtained (data not shown). The purity of the isolated cells was estimated by flow cytometric analysis (with FACS Calibur, Becton Dickinson, Mountain View, Calif.). Morphological analysis was performed after staining with May-Grunewald/Giemsa solution. The purity of B-CLL and B-PLL cells was >98%.

Incubation of Intact B-CLL cells:

10×10⁶ cells were suspended in 1 mL PBS and pre-incubated for two minutes with/without azodicarboxylic acid bis(dimethylamide), abbreviated diamide, (100 μM) prior to stimulation with arachidonic acid (40 μM) and/or calcium ionophore A23187 (1 μM). The cells were stimulated for five minutes at 37° C. and the incubations were terminated with 1 mL methanol.

### Incubation of Sonicated B-CLL Cells:

10×10⁶ cells were resuspended in 1 ml calcium-free PBS including EDTA (2 mM) and sonicated 3×5 s. The cells were pre-incubated for two minutes in the presence of ATP (1 mM) prior to addition of calcium chloride (2 mM) and arachidonic acid (40 μM). The reaction was terminated with 1 mL methanol after five minutes of incubation at 37° C.

### Analysis of Leukotrienes:

After addition of 0.5 ml PBS and the internal standard PGB₁ (100 pmol) to the samples, the cells were centrifuged (2500 rpm, 5 min). The supernatant was subsequently subjected to solid phase extraction on Chromabond C₁₈ columns (200 mg, Macherey & Nagel). The methanol eluate was analysed on Waters Alliance 2695 reverse phase HPLC and detected with Waters PDA 996. Methanol:water:trifiuoroacetic acid (70:30:0.007, v/v) was used as mobile phase at a flow rate of 1.2 mL/min. Qualitative analysis was performed by comparison of retention times of synthetic standards and quantitative determinations were performed by computerized integration of the area of eluted peaks.

### Expression of BLT1:

Fresh blood samples from normal donors and fresh samples from patients were Ficoll-Isopaque separated and washed in PBS. For analysis of whole blood leukocytes (including granulocytes) from healthy donors, cells were washed in PBS and lysed with FACS lysing solution (Becton Dickinson) is and washed in PBS. Frozen patient samples (B-CLL and B-PLL) were thawed (as described above) and washed in PBS. After resuspending cells in 100 μL PBS, antibodies were added according to manufacturer's instructions and incubated at room temperature for 10 minutes. The cells were washed in 2 mL PBS and fixed in 1% paraformaldehyde, before analysis with FACS Calibur (Becton Dickinson) using the CeliQuest software.

In this study all the antibodies used for flow cytometry were directly conjugated with either fluorescein isothionine (FITC), phycoerythrin (Pe) or peridinin chlorophyll protein (PerCP).

The BLT1 antibody 7B1 FITC was raised in-house (see: Biochem. Biophys. Res. Commun. 279, 520 (2000)). IgG1-FITC, IgG1-Pe, IgG1 Percp, CD4-Pe, CD5-Pe, CD8-Percp, CD14-FITC, CD14-Pe, CD19-FITC, CD19-Pe, CD20-Percp, CD22-Pe, CD33-FITC, CD33-Pe, IgG2a-FITC (Becton Dickinson).

DNA Synthesis:

Purified B-CLL cells were cultured in RPMI 1640 medium, supplemented with 10% FCS, 2 mM L-glutamine, 100 U/mL penicillin and 100 μg/mL streptomycin and incubated at 3720 C. in an atmosphere of 5% CO₂. 2×10⁵ of B-CLL cells were seeded in 200 μL medium in 96-well plates. B-CLL cells were pretreated with MK-886 (a specific FLAP inhibitor) (10⁻⁶ to 10⁻⁹ M) or BWA4C (a specific 5-LO inhibitor) (10⁻⁷ to 10⁻⁹ M) for 30 min, before co-culturing with irradiated (15,000 Rad) CD40L expressing L (CD40L-L) cells or control L (L) cells in the presence of inhibitors. LTB₄ (10⁻⁷ M) was present in the indicated cultures. Each sample was represented by triplicates. 1 μCi ³H-thymidine was present in the wells for the final eight 15 hours of the 96 hr cultures. The cells were harvested onto glass fibre filter and radioactivity was measured in a liquid scintillation counter.

### Flow Cytometry Analysis of CD23, CD54 and CD150 Expression:

Cultured (described above) B-CLL cells were collected (without the plastic attached L cells) and used for FACS detection. Surface marker expression was detected by indirect immunofluorescence. One million cells/sample were washed in cold PBS containing 1% FCS and 0.1% sodium azide and then exposed to the relevant antibodies. The cells were washed and incubated with the RPE conjugated secondary antibody. All incubations were done at 4° C.

Samples were run on a Becton Dickinson FACScan flow cytometer (Becton Dickinson, Mountain View, Calif.). The CellQuest software (Becton Dickinson) was used both for acquisition and analysis of the samples. Ten thousand events were collected on a FACScan flow cytometer, and the results were analysed using CellQuest (Becton Dickinson) software.

Only the viable cells were considered for analysis based on their light scatter (FSC/SSC) characteristics. The following antibodies were used: MAb MHM-6 (anti-CD23, from Dr. M. Rowe, University of Wales, Cardiff, Wales, UK), MAb LB-2 (anti-CD54, from E. A. Clark, University of Washington, Seattle, Wash.), MAb IPO-3 (anti-SLAM, kind gift from S. Sidorenko, Acad. of Science of Ukraine, Kiev, Ukraine) and RPE conjugated rabbit anti-mouse Ig F(ab′)₂ (Dako, Copenhagen, Denmark) were used as secondary antibody.

### Results

Biosynthesis of Leukotrienes in B-CLL cells:

The capacity of B-CLL cells to produce leukotrienes was investigated. The cells were challenged with either calcium ionophore A23187, arachidonic acid or calcium ionophore A23187 plus arachidonic acid. No cell clones produced detectable amounts of leukotrienes after challenge with either calcium ionophore A23187 or arachidonic acid only. Activation of the cells with calcium ionophore A23187 and arachidonic acid led to the formation of LTB₄ (mean 2.6±0.8 pmol/10⁶ cells). Preincubation of intact cells with the thiol-reactive agent diamide, prior to addition of calcium ionophore and arachidonic acid, led to a markedly increased production of LTB₄ (mean 33.5±1.2 pmol/10⁶ cells) in comparison to untreated intact cells (FIG. 1). These results are in agreement with earlier reports (see Proc. Natl. Acad. Sci. USA 89, 3521 (1992) and Eur. J. Biochem. 242, 90 (1996)). Similar amounts of LTB₄ (mean 34.8±1.7 pmol/10⁶ cells) were produced in broken-cell preparation, incubated with arachidonic acid. There was no obvious correlation between the capacity to produce leukotrienes and the clinical stage of the disease (data not shown). Taken together, the results demonstrated that all investigated B-CLL clones had the capacity to produce LTB₄ and that all B-CLL clones contained substantial amounts of 5-lipoxygenase which could be activated under certain conditions.

### BLT1 Expression:

Peripheral blood leukocytes from healthy donors were analysed with FACS for the expression of BLTR1. Gates for granulocytes, lymphocytes and monocytes were set on the basis of forward and side scatter. Virtually all cells gated as granulocytes (and CD33 positive) expressed BLT1 (FIG. 2a). Cells in the monocyte gate (CD14 positive) showed the same pattern of BLT1 expression (data not shown). In the lymphocyte gate, no expression of BTL1 was observed on peripheral non-activated CD4⁺- or CD8⁺-positive T-lymphocytes (FIGS. 2b and 2c). These results are in agreement with the observation that naive non-activated mouse T lymphocytes do not express BLT1 (see Nat. Immunol. 4, 982 (2003)). In contrast, 30-50% of CD19, CD20 and CD22 expressing peripheral B-lymphocytes stained positively for BLT1 (FIG. 2d). The BLT1 expression on peripheral B-lymphocytes was weaker than on granulocytes and monocytes and showed a pattern of gradually increased expression within the peripheral B-lymphocyte population. Similar results have recently been reported (see Int. Immunopharmacol. 3, 1467 (2003)).

B-cells from five patients with B-CLL and two with B-prolymphocytic leukemia (B-PLL) were analysed with FACS for BLT1 expression. BLT1 expression analysed with FACS varied from about 15% to 85% in 5 B-CLL clones (average 42%) (FIG. 2e). In the B-PLL group, the average expression of BLT1 was 74% in the two investigated clones. (FIG. 2f).

### Effects of Leukotriene Synthesis Inhibitors on DNA Synthesis in B-CLL Cells:

In order to elucidate if leukotrienes are of importance for proliferation of B-CLL, the cells were cultivated in the presence of leukotriene biosynthesis inhibitors. B-CLL cells were co-cultured with CD40L expressing L cells or control L cells for 96 hr in the absence or presence of MK-886 (a specific FLAP inhibitor) or BWA4C (a specific 5-lipoxygenase inhibitor). CD40-CD40L interactions activated B-CLL cells and resulted in an increased DNA synthesis, measured as ³H-thymidin incorporation during the final eight hours of four days cultures (FIG. 3). MK-886, at a concentration of 100 nM, markedly inhibited DNA synthesis induced by CD40-ligand stimulation (FIG. 3A). Due to the relatively high binding of MK-886 to serum proteins (see Can. J. Physiol. Pharmacol. 67, 456 (1989)), the effect of 1 μM MK-886 on DNA synthesis was also investigated in certain experiments. This concentration of the inhibitor only caused a little more pronounced inhibition of DNA synthesis. The inhibitory action of 1 μM and 100 nM MK-886 on thymidine incorporation was 46 and 38%, respectively. Leukotriene B₄ (final concentration 150 nM) did not amplify CD40-induced thymidine incorporation. However, exogenously added LTB₄ (150 nM) almost completely reversed the inhibitory effect of MK-886 on thymidine incorporation. The specific 5-lipoxygenase inhibitor BWA4C was an even more potent inhibitor than MK-886 to block DNA synthesis (FIG. 3B). A significant inhibitory effect of BWA4C on thymidine incorporation was observed at 10 nM. In line with the results with MK-866, exogenous addition of LTB₄ (150 nM) almost completely reversed the inhibitory action of 100 nM BWA4C on thymidine incorporation (FIG. 3B). The cell survival after four days cultivation was about 80% in all B-CLL cultures stimulated with CD40L-L, both in the absence or presence of inhibitor or LTB₄ (data not shown). Taken together, these specific inhibitors of leukotriene synthesis caused at low concentrations a pronounced inhibition of DNA synthesis, which could be reversed by exogenous addition of LTB₄.

### Effects of Leukotriene Biosynthesis Inhibitors and LTB₄ on CD23, CD54 and CD150 Expression in B-CLL Cells:

The expression of CD23 is a marker of activation of B-cells. CD54 (ICAM-1) is an important adhesive molecule expressed to various extents on many B-CLL clones. CD150 is an antigen involved in the bidirectional stimulation of T- and B-cells and is upregulated on activated B-cells. FACS analysis demonstrated that CD40-CD40L interactions caused an increased expression of all three antigens (FIG. 4). MK-886 and BWA4C, at a concentration of 100 nM, markedly counteracted this CD40-induced increased expression of CD23, CD54 and CD150. Leukotriene B₄ did not cause any significant effect alone on the expression of the investigated is antigens. However, addition of 150 nM LTB₄ almost completely reversed the inhibitory effect of the inhibitors on antigen expression (FIG. 4). These results show that LTB₄ is involved in the expression of these antigens, which are associated with activation and tissue infiltration of B-CLL cells.

1-12. (canceled)

13. A method of treating B-CLL, B-PLL or B-cell lymphoma, which method comprises administering an inhibitor of the biosynthesis and/or function of LTB₄ to a patient in need of such treatment.

14. A method as claimed in claim 13, wherein the inhibitor of the function of LTB₄ is an antagonist of the BLT) receptor.

15. A method as claimed in claim 13, wherein the inhibitor of the biosynthesis of LTB₄ is an inhibitor of 5-LO, and inhibitor of FLAP and/or an inhibitor of LTA₄ hydrolase.

16. A method as claimed in claim 13, wherein the method comprises administering to the patient an inhibitor of 5-LO or an inhibitor of FLAP.

17. A method as claimed in claim 16, wherein the inhibitor of 5-LO is BWA4C, or the inhibitor of FLAP is MK-886.

18. A method as claimed in claim 13, wherein the inhibitor of the biosynthesis and/or function of LTB₄ is the sole cancer chemotherapeutic agent administered to the patient.

19. A method as claimed in claim 18, wherein the inhibitor of the function of LTB₄ is an antagonist of the BLT1 receptor.

20. A method as claimed in claim 18, wherein the inhibitor of the biosynthesis of LTB₄ is an inhibitor of 5-LO, and inhibitor of FLAP and/or an inhibitor of LTA₄ hydrolase.

21. A method as claimed in claim 18, wherein the method comprises administering to the patient an inhibitor of 5-LO or an inhibitor of FLAP.

22. A method as claimed in claim 21, wherein the inhibitor of 5-LO is BWA4C, or the inhibitor of FLAP is MK-886.

23. A method of treating B-CLL, B-PLL or B-cell lymphoma, which method comprises administering an inhibitor of the biosynthesis and/or function of LTB₄ to a patient in need of such treatment, which patient is administered a cancer chemotherapeutic agent having a different mechanism of action.

24. A method as claimed in claim 13, wherein the patient is human.

25. A combination product comprising:
(A) an inhibitor of the biosynthesis and/or function of LTB4, or a pharmaceutically-acceptable derivative thereof; and
(B) a cancer chemotherapeutic agent having a different mechanism of action, or a pharmaceutically acceptable derivative thereof,
wherein each of components (A) and (B) is formulated in admixture with a pharmaceutically-acceptable adjuvant, diluent or carrier.
